(19) United States
(12) Patent Application Publication
(10) Pub. No.: US 2023/0098254 A1
(43) Pub. Date: Mar. 30, 2023

(54) SCUTELLARIAE RADIX COMPOUNDS AND USE THEREOF FOR INHIBITING OXIDATIVE PHOSPHORYLATION PATHWAY OF MITOCHONDRIA

(71) Applicant: NANJING SHIJIANG MEDICINE TECHNOLOGY CO., LTD, Jiangbei New District Nanjing, Jiangsu (CN)

(72) Inventors: Yufeng SHI, Jiangsu (CN); Wenjiang MA, Jiangbei New District Nanjing, Jiangsu (CN)

(73) Assignee: NANJING SHIJIANG MEDICINE TECHNOLOGY CO., LTD, Jiangbei New District Nanjing, Jiangsu (CN)

(21) Appl. No.: 17/909,422

(22) PCT Filed: Mar. 8, 2021
(86) PCT No.: PCT/CN2021/079602

§ 371 Date: Sep. 5, 2022

(30) Foreign Application Priority Data
Mar. 6, 2020 (CN) 202010153303.2

(51) Int. Cl. A61K 31/352; A61K 45/06

(52) U.S. Cl. CPC A61K 31/352; A61K 45/06

(57) ABSTRACT

Provided are scutellariae radix compounds and the use thereof for inhibiting the oxidative phosphorylation pathway of mitochondria. Specifically provided is the use of a compound of formula I, or an optical isomer thereof, or a racemate thereof, or a solvate thereof, or a pharmaceutically acceptable salt thereof in the preparation of a composition or a preparation. The composition or the preparation is used for one or more uses selected from the group consisting of: (a) inhibiting the oxidative phosphorylation pathway of mitochondria; (b) preventing and/or treating diseases associated with the oxidative phosphorylation pathway of mitochondria; and (c) preventing and/or treating cancers. The compounds can be used for inhibiting the oxidative phosphorylation pathway of mitochondria and preventing and/or treating diseases associated with the oxidative phosphorylation pathway of mitochondria, and especially have a significant inhibitory effect on the upregulation of the oxidative phosphorylation pathway of mitochondria or tumor cells with a low mPTP activity.

### FIELD OF THE INVENTION

The present invention relates to the field of medicine. Specifically, the present invention relates to a scutellariae radix compounds and use thereof for inhibiting oxidative phosphorylation pathway of mitochondria.

### BACKGROUND TECHNOLOGY

Mitochondria is ubiquitous in eukaryotic cells, providing energy for the activities of cells and other intermediate products necessary for cells growth. Mitochondria, as the energy factory in cell, is an indispensable organelle for tumorigenesis of tumor cell, and mitochondria reprogramming is also one of the hallmark features of tumor. In recent years, anti-tumor agents targeting, mitochondria have become the frontier of tumor biology research and the hot spot of anti-tumor drug research and development, especially the highly recurrent malignant tumors that cannot be solved by traditional radiotherapy, chemotherapy and other treatment methods are extremely dependent on mitochondria function. The development of drugs that can target the mitochondria of tumor cells is expected to provide an effective treatment for malignant tumors and effectively prolong the survival period of patients.

Oxidative Phosphorylation (OXPHOS) is one of the most important pathways in mitochondria, which utilizes NADH and FADH derived from pathways such as the tricarboxylic acid cycle and fat oxidation to produce ATP. The oxidative phosphorylation pathway of mitochondria is composed of more than 90 proteins, which form five protein complexes, complexes I, II, III, IV and V, respectively. The first four protein complexes (complexes I, II, III and IV), also known as the electron transport chain, receive electrons from electron donors NADH and FADH and transfer them to oxygen. In the process of electron transfer, hydrogen ions are pumped from the mitochondrial inner membrane to the intermembrane space between the mitochondrial inner membrane and the mitochondrial outer membrane, thereby forming a hydrogen ion gradient and potential difference inside and outside the inner membrane. The energy stored in the mitochondria membrane potential drives complex V in the oxidative phosphorylation pathway to generate ATP. In recent years, tumor research and large patient data have shown that inhibitor of the oxidative phosphorylation pathway of mitochondria can effectively inhibit tumor growth. However, the current inhibitor of the oxidative phosphorylation pathway of mitochondria is weak or have no effect in tumor treatment; or it is highly toxic and cannot effectively distinguish normal cells from tumor cells, so it has great side effects and cannot be developed as an anticancer drug.

The oxidative phosphorylation pathway of mitochondria is regulated by the mitochondria permeability transition pore (mPTP). When the mitochondria permeability transition pore is opened, the mitochondria membrane potential difference decreases, and the by-product of the oxidative phosphorylation pathway, peroxide ROS, is discharged from the mitochondria. Studies have shown that the mitochondria permeability transition pore is inactive in some tumor cells, especially in highly malignant tumor cells with stem cell properties, while it is active in normal cells. mPTP-dependent mitochondrial inhibitors can effectively inhibit mitochondrial function in the case that the mitochondria permeability transition pore is inactive, while such inhibitors lose the inhibitory effect in the case that the channel is active. Therefore, such mitochondrial inhibitors can effectively distinguish normal cells from tumor cells, and can effectively inhibit the growth of tumors without obvious damage to normal cells. In particular, the highly recurrent malignant tumor cells that cannot be removed by conventional radiotherapy, chemotherapy and other treatment methods are extremely dependent on this pathway, and specific targeted inhibition of this pathway can effectively kill the currently untreatable malignant tumors that are easy to recur or metastasize in the clinic at present, while it has little side effects on normal cells and has a strong druggability, and can be developed into the relatively rare malignant tumor-targeted drugs at present, which provides a solution for the urgent clinical need that has not been met.

Therefore, there is a need in the art to develop a drug that can safely and effectively inhibit the oxidative phosphorylation pathway of mitochondria, the drug can effectively inhibit tumor cells, especially highly malignant tumors that cannot be effectively treated by conventional radiotherapy and chemotherapy and have a large possibility of recurrence and metastasis, and the drug has fewer side effects on normal cells and is relatively safe to use.

### SUMMARY OF THE INVENTION

It is an object of the present invention to provide a compound that can safely and effectively inhibit oxidative phosphorylation pathway of mitochondria and the use thereof.

In the first aspect of the present invention, it provides a use of a compound of formula I, or an optical isomer thereof, or a racemate thereof, or a solvate thereof, or a pharmaceutically acceptable salt thereof in the preparation of a composition or a preparation, the composition or the preparation is used for one or more uses selected from the group consisting of: (a) inhibiting the oxidative phosphorylation pathway of mitochondria; (b) preventing and/or treating diseases associated with oxidative phosphorylation pathway of mitochondria; and (c) preventing and/or treating cancers;

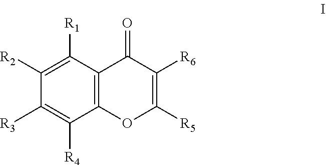

wherein,

R₁, R₂, R₃, R₄, R₅ and R₆ are each independently hydrogen, halogen, —CN, hydroxyl sulfhydryl, nitro, amino, —COOH, —CHO, substituted or unsubstituted C1-C12 alkyl, substituted or unsubstituted C3-C16 cycloalkyl, substituted or unsubstituted C1-C12 alkoxyl, substituted or unsubstituted C1-C12 alkylthio, substituted or unsubstituted 3-16 membered heterocycloalkyl, substituted or unsubstituted C6-C16 aryl, substituted or unsubstituted 3-16 membered heteroaryl, substituted or unsubstituted glycosyl-O—, substituted or unsubstituted glycosyl-S—, substituted or unsubstituted uronic acid group-O—, or substituted or unsubstituted uronic acid group-S—;

each “substituted” means that one or more (preferably 1, 2, 3, or 4) hydrogen atoms on the group are substituted by a substituent selected from the group consisting of C1-C8 alkyl, C3-C8 cycloalkyl, C1-C8 haloalkyl, C3-C8 halocycloalkyl, halogen, nitro, —CN, hydroxyl, sulfhydryl, amino, C1-C4 carboxyl, C2-C4 ester group, C2-C4 amido, C1-C8 alkoxyl, C1-C8 alkylthio, C1-C8 haloalkoxyl, C1-C8 haloalkylthio, C6-C12 aryl, 5-10 membered heteroaryl, 5-10 membered heterocycloalkyl;

the heterocyclic ring of the heterocycloalkyl and heteroaryl each independently contains 1-4 (preferably 1, 2, 3 or 4) heteroatoms selected from the group consisting of N, O and S.

In another preferred embodiment, the “substituted” means that one or more (preferably 1, 2, 3, or 4) hydrogen atoms on the group are substituted by a substituent selected from the group consisting of C1-C6 alkyl, C3-C8 cycloalkyl, C1-C6 haloalkyl, C3-C8 halocycloalkyl, halogen, nitro, —CN, hydroxyl, sulfhydryl, amino, C1-C4 carboxyl, C2-C4 ester group, C2-C4 amido, C1-C6 alkoxyl, C1-C6 alkylthio, C1-C6 haloalkoxyl, C1-C6 haloalkylthio, C6-C12 aryl, 5-10 membered heteroaryl, 5-10 membered heterocycloalkyl.

In another preferred embodiment, the “substituted” means that one or more (preferably 1, 2, 3, or 4) hydrogen atoms on the group are substituted by a substituent selected from the group consisting of C1-C4 alkyl, C3-C8 cycloalkyl, C1-C4 haloalkyl, C3-C8 halocycloalkyl, halogen, nitro, —CN, hydroxyl, sulfhydryl, amino, C1-C4 carboxyl, C2-C4 ester group, C2-C4 amido, C1-C4 alkoxyl, C1-C4 alkylthio, C1-C4 haloalkoxyl, C1-C4 haloalkylthio, C6-C12 aryl, 5-10 membered heteroaryl, 5-10 membered heterocycloalkyl.

In another preferred embodiment, the heterocyclic ring of the heterocycloalkyl and heteroaryl each independently contains 1-4 (preferably 1, 2, 3 or 4) heteroatoms selected from the group consisting of N, O and S.

In another preferred embodiment, R₁, R₂, R₃, R₄, R₅ and R₆ are each independently hydrogen, halogen, —CN, hydroxyl, sulfhydryl, nitro, amino, —COOH, —CHO, substituted or unsubstituted C1-C10 alkyl, substituted or unsubstituted C3-C10 cycloalkyl, substituted or unsubstituted C1-C10 alkoxyl, substituted or unsubstituted C1-C10 alkylthio, substituted or unsubstituted 3-12 membered heterocycloalkyl, substituted or unsubstituted C6-C16 aryl, substituted or unsubstituted 3-12 membered heteroaryl, substituted or unsubstituted glycosyl-O—, substituted or unsubstituted glycosyl-S—, substituted or unsubstituted uronic acid group-O—, or substituted or unsubstituted uronic acid group-S—.

In another preferred embodiment, R₁, R₂, R₃, R₄, R₅ and R₆ are each independently hydrogen, halogen, —CN, hydroxyl, sulfhydryl, nitro, amino, —COOH, —CHO, substituted or unsubstituted C1-C6 alkyl, substituted or unsubstituted C3-C8 cycloalkyl, substituted or unsubstituted C1-C6 alkoxyl, substituted or unsubstituted C1-C6 alkylthio, substituted or unsubstituted 3-10 membered heterocycloalkyl, substituted or unsubstituted C6-C12 aryl, substituted or unsubstituted 3-10 membered heteroaryl, substituted or unsubstituted glycosyl-O—, substituted or unsubstituted glycosyl-S—, substituted or unsubstituted uronic acid group-O—, or substituted or unsubstituted uronic acid group-S—.

In another preferred embodiment, R₁, R₂, R₃, R₄, R₅ and R₆ are each independently hydrogen, halogen, —CN, hydroxyl, nitro, amino, —COOH, —CHO, substituted or unsubstituted C1-C6 alkyl, substituted or unsubstituted C3-C8 cycloalkyl, substituted or unsubstituted C1-C4 alkoxyl, substituted or unsubstituted C1-C4 alkylthio, substituted or unsubstituted 3-10 membered heterocycloalkyl, substituted or unsubstituted C6-C12 aryl, substituted or unsubstituted 3-10 membered heteroaryl, substituted or unsubstituted glycosyl-O—, substituted or unsubstituted glycosyl-S—, substituted or unsubstituted acid group-O—, or substituted or unsubstituted uronic acid group-S—.

In another preferred embodiment, R₁, R₂, R₃, R₄, R₅ and R₆ are each independently hydrogen, halogen, —CN, hydroxyl, sulfhydryl, nitro, amino, —COOH, —CHO, substituted or unsubstituted C1-C6 alkyl, substituted or unsubstituted C3-C8 cycloalkyl, substituted or unsubstituted C1-C4 alkoxyl, substituted or unsubstituted C1-C4 alkylthio, substituted or unsubstituted 3-10 membered heterocycloalkyl, substituted or unsubstituted C6-C8 aryl, substituted or unsubstituted 3-8 membered heteroaryl, substituted or unsubstituted glycosyl-O—, substituted or unsubstituted glycosyl-S—, substituted or unsubstituted uronic acid group-O—, or substituted or unsubstituted uronic acid group-S—.

In another preferred embodiment, R₁ and R₂ are each independently hydrogen, hydroxyl or sulfhydryl.

In another preferred embodiment, R₁ and R₂ are each independently hydrogen and hydroxyl.

In another preferred embodiment, R₃ is hydroxyl, sulfhydryl, substituted or unsubstituted C1-C6 alkoxyl, substituted or unsubstituted C1-C6 alkylthio, substituted or unsubstituted glycosyl-O—, substituted or unsubstituted glycosyl-S—, substituted or unsubstituted uronic acid group-O—, or substituted or unsubstituted uronic acid group-S—.

In another preferred embodiment, the glycosyl is monoglycosyl, bisglycosyl, oligoglycosyl or polyglycosyl.

In another preferred embodiment, the uronic acid group is monouronic acid group, bisuronic acid group, oligouronic acid group, polysuronic acid group.

In another preferred embodiment, R₃ is hydroxyl, sulfhydryl, substituted or unsubstituted C1-C6 alkoxyl, substituted or unsubstituted C1-C6 alkylthio, substituted or unsubstituted monoglycosyl-O—, substituted or unsubstituted monoglycosyl-S—, substituted or unsubstituted monouronic acid group-O—, substituted or unsubstituted monouronic acid group-S—, substituted or unsubstituted bisglycosyl-O—, substituted or unsubstituted bisglycosyl-S—, substituted or unsubstituted bisuronic acid group-O—, or substituted or unsubstituted bisuronic acid group-S—.

In another preferred embodiment, the monoglycose contains 3-6 carbon atoms.

In another preferred embodiment, the monoglycose is L-monoglycose or D-monoglycose.

In another preferred embodiment, the monoglycose is pentose or hexose.

In another preferred embodiment, the monoglycose is pyranose or furanose.

In another preferred embodiment, the monoglycose is glyceraldehyde, erythrose, threose, arabinose, ribose, xylose, lyxose, glucose, mannose, fructose or galactose.

In another preferred embodiment, the glucosyl is L-glucosyl or D-glucosyl.

In another preferred embodiment, the bisglycose is maltose, lactose, sucrose or trehalose.

In another preferred embodiment, the oligoglycose is cyclodextrin.

In another preferred embodiment, the oligoglycose is formed by linking 3-9 monoglycoses through glycosidic bonds.

In another preferred embodiment, the uronic acid group is glucuronic acid group, galacturonic acid group, mannuronic acid group, iduronic acid group or guluronic acid group.

In another preferred embodiment, the glucuronic acid group is L-glucuronic acid group or D-glucuronic acid group.

In another preferred embodiment, R₃ is hydroxyl, sulfhydryl, substituted or unsubstituted C1-C4 alkoxyl, substituted or unsubstituted C1-C4 glyceraldehyde group-O—, erythrosyl-O—, threosyl-O—, arabinosyl-O—, ribosyl-O—, xylosyl-O—, lyxosyl-O—, glucosyl-O—, mannosyl-O—, fructosyl-O—, galactosyl-O—, maltosyl-O—, lactosyl-O—, sucrosyl-O—, trehalosyl-O—, glucuronic acid group-O—, galacturonic acid group-O—, mannuronic acid group-O—, iduronic acid group-O—, guluronic acid group-O—, glyceraldehyde group-S—, erythrosyl-S—, threosyl-S—, arabinosyl-S—, xylosyl-S—, lyxosyl-S—, glucosyl-S—, mannosyl-S—, fructosyl-S—, galactosyl-S—, maltosyl-S—, lactosyl-S—, sucrosyl-S—, trehalosyl-S—, glucuronic acid group-S—, galacturonic acid group-S—, mannuronic acid group-S—, iduronic acid group-S— or guluronic acid group-S—.

In another preferred embodiment, R₃ is hydroxyl, sulfhydryl, substituted or unsubstituted C1-C4 alkoxyl, substituted or unsubstituted C1-C4 alkylthio, glucosyl-—, glucuronic acid group-O—, galacturonic acid group-O—, mannuronic acid group-O—, iduronic acid group-O— or guluronic acid group-O—.

In another preferred embodiment, R₃ is hydroxyl, sulfhydryl, methoxyl, methylthio, or

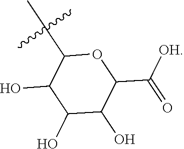 [SMILES: *C1OC(C(=O)O)C(O)C(O)C1O]

In another preferred embodiment, R₃ is hydroxyl, sulfhydryl, methoxyl, methylthio, or

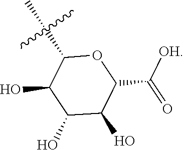

In another preferred embodiment, R₄ is hydrogen, halogen, —CN, hydroxyl, sulfhydryl, nitro, amino, —COOH, —CHO, substituted or unsubstituted C1-C6 alkyl, substituted or unsubstituted C3-C8 cycloalkyl, substituted or unsubstituted C1-C8 alkoxyl, substituted or unsubstituted C1-C8 alkylthio.

In another preferred embodiment, R₄ is hydrogen, halogen, —CN, hydroxyl, sulfhydryl, nitro, amino, —COOH, —CHO, substituted or unsubstituted C1-C6 alkyl, substituted or unsubstituted C3-C8 cycloalkyl, substituted or unsubstituted C1-C8 alkoxyl, substituted or unsubstituted C1-C8 alkylthio.

In another preferred embodiment, R₄ is hydrogen, halogen, —CN, hydroxyl, sulfhydryl, nitro, amino, —COOH, —CHO, substituted or unsubstituted C1-C6 alkyl, substituted or unsubstituted C3-C8 cycloalkyl, substituted or unsubstituted C1-C4 alkoxyl, substituted or unsubstituted C1-C4 alkylthio.

In another preferred embodiment, R₄ is hydrogen, methoxyl, or methylthio.

In another preferred embodiment, R₅ is substituted or unsubstituted C₆-C16 aryl, or substituted or unsubstituted 3-16 membered heteroaryl.

In another preferred embodiment, R₅ is substituted or unsubstituted C6-C12 aryl, or substituted or unsubstituted 3-12 membered heteroaryl.

In another preferred embodiment, R₅ is substituted or unsubstituted C6-C10 aryl, or substituted or unsubstituted 3-10 membered heteroaryl.

In another preferred embodiment, R₅ is substituted or unsubstituted C6-C8 aryl, or substituted or unsubstituted 3-8 membered heteroaryl.

In another preferred embodiment, R₅ is substituted or unsubstituted C6 aryl, substituted or unsubstituted C7 aryl, substituted or unsubstituted C8 aryl, substituted or unsubstituted C9 aryl, substituted or unsubstituted C10 aryl, substituted or unsubstituted C11 aryl, substituted or unsubstituted C12 aryl, substituted or unsubstituted 4 membered heteroaryl, substituted or unsubstituted 5 membered heteroaryl, substituted or unsubstituted 6 membered heteroaryl, substituted or unsubstituted 7 membered heteroaryl, substituted or unsubstituted 8 membered heteroaryl, substituted or unsubstituted 9 membered heteroaryl, substituted or unsubstituted 10 membered heteroaryl, substituted or unsubstituted 11 membered heteroaryl, substituted or unsubstituted 2 membered heteroaryl.

In another preferred embodiment, R₅ is substituted or unsubstituted phenyl, or substituted or unsubstituted naphthyl.

In another preferred embodiment, R₅ is substituted or unsubstituted phenyl.

In another preferred embodiment, R₅ is substituted or unsubstituted phenyl, and the “substituted” means that one or more (preferably 1, 2, 3, or 4) hydrogen atoms on the phenyl are substituted by a substituent selected from the group consisting of hydroxyl, sulfhydryl.

In another preferred embodiment, R₅ is substituted or unsubstituted phenyl, and the “substituted” means that one or two hydrogen atoms on the phenyl are substituted by a substituent selected from the group consisting of hydroxyl, sulfhydryl.

In another preferred embodiment, R₅ is substituted or unsubstituted phenyl, and the “substituted” means that one para-hydrogen on the phenyl is substituted by a substituent selected from the group consisting of hydroxyl and sulfhydryl.

In another preferred embodiment, R₅ is substituted or unsubstituted phenyl, and the “substituted” means that one meta-hydrogen and one para-hydrogen on the phenyl are substituted by a substituent selected from the group consisting of hydroxyl and sulfhydryl.

In another preferred embodiment, R₅ is substituted or unsubstituted phenyl, and the “substituted” means that two ortho-hydrogens on the phenyl are substituted by a substituent selected from the group consisting of hydroxyl and sulfhydryl.

In another preferred embodiment, R₅ is

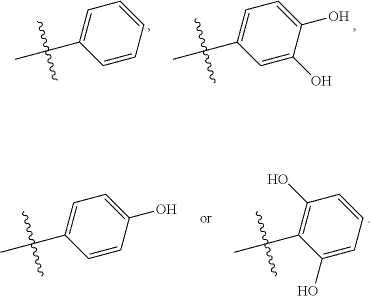 [SMILES: *c1ccccc1 ; *c1ccc(O)c(O)c1 ; *c1ccc(O)cc1 ; *c1c(O)cccc1O]

In another preferred embodiment, R₆ is hydrogen, halogen, —CN, hydroxyl, sulfhydryl, nitro, amino, —COOH, —CHO, substituted or unsubstituted C1-C6 alkyl, substituted or unsubstituted C3-C8 cycloalkyl.

In another preferred embodiment, R₆ is hydrogen, hydroxyl or sulfhydryl.

In another preferred embodiment, the compound of formula I is selected from the following group.

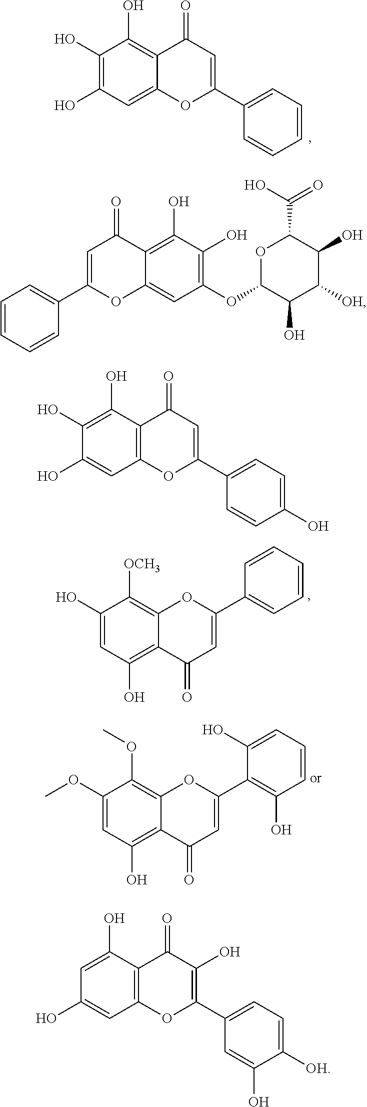

In another preferred embodiment, the pharmaceutically acceptable salt of the compound of formula I is a salt formed by the compound of formula I and an acid selected from the group consisting of hydrochloric acid, mucic acid, D-glucuronic acid, hydrobromic acid, hydrofluoric acid, sulfuric acid, nitric acid, phosphoric acid, formic acid, acetic acid, propionic acid, oxalic acid, malonic acid, succinic acid, fumaric acid, maleic acid, lactic acid, malic acid, tartaric acid, citric acid, picric acid, methanesulfonic acid, benzenemethanesulfonic acid, benzenesulfonic acid, aspartic acid, glutamic acid, and combinations thereof.

In another preferred embodiment, the diseases associated with oxidative phosphorylation pathway of mitochondria are selected from the group consisting of cancers, immune related diseases, neurodegenerative diseases, viral infection and/or its related diseases, and combinations thereof.

In another preferred embodiment, the diseases associated with oxidative phosphorylation pathway of mitochondria are diseases associated with the upregulation of the oxidative phosphorylation pathway of mitochondria.

In another preferred embodiment, preventing and/or treating diseases associated with oxidative phosphorylation pathway of mitochondria is achieved by (i) inhibiting the activity of the oxidative phosphorylation pathway of mitochondria to prevent and/or treat diseases associated with oxidative phosphorylation pathway of mitochondria.

In another preferred embodiment, the cancer is selected from the group consisting of lung cancer, pancreatic cancer, breast cancer, lymphoma, prostate cancer, brain cancer, leukemia, liver cancer, melanoma, intestinal cancer, kidney cancer, and combinations thereof.

In another preferred embodiment, the cancer is selected from the group consisting of adenocarcinoma, ductal carcinoma, squamous cell carcinoma, and combinations thereof.

In another preferred embodiment, the cancer is lowly differentiated, moderately differentiated or highly differentiated cancer cell.

In another preferred embodiment, the lowly differentiated cancer cell means that the ratio (L1/L2) of the differentiation degree L1 of the cancer cell to the differentiation degree L2 of normal tissue cell is ≤0.8, preferably ≤0.7, more preferably ≤0.6, more preferably ≤0.5, more preferably ≤0.4, more preferably ≤0.3, more preferably ≤0.2, more preferably ≤0.1, most preferably ≤0.05.

In another preferred embodiment, the moderately differentiated cancer cell means that the ratio (M1/M2) of the differentiation degree M1 of the cancer cell to the differentiation degree M2 of normal tissue cell is 0.2-0.8, preferably 0.3-0.7, more preferably 0.4-0.6, most preferably 0.45-0.55.

In another preferred embodiment, the highly differentiated cancer cell means the ratio (H1/H2) of the differentiation degree H1 of cancer cell to the differentiation degree H2 of normal tissue cell is 0.7-1.3, preferably 0.8-1.2, more preferably 0.9-1.1, most preferably 0.95-1.05.

In another preferred embodiment, the mitochondria permeability transition pore of the cancer cell is low activity.

In another preferred embodiment, the low activity of the mitochondria permeability transition pore means that the ratio (A1/A0) of the activity level or expression level A1 of the mitochondria permeability transition pore in a cell (such as the cancer cell) to the activity level or expression level A0 of the mitochondria permeability transition pore in a normal cell (the same type of cell) is ≤0.8, preferably ≤0.7, more preferably ≤0.6, more preferably ≤0.5, more preferably ≤0.4, more preferably ≤0.3, more preferably ≤0.2, more preferably ≤0.1, most preferably ≤0.05.

In another preferred embodiment, the cancer is less differentiated cancer.

In another preferred embodiment, the less differentiated cancer means that the ratio (D1/D2) of differentiation degree D1 of the cancer cell to the differentiation degree D2 of normal tissue cell is ≤0.8, preferably ≤0.7, more preferably ≤0.6, more preferably ≤0.5, more preferably ≤0.4, more preferably ≤0.3, more preferably ≤0.2, more preferably ≤0.1, and most preferably ≤0.05.

In another preferred embodiment, the cancer is not sensitive to conventional radiotherapy and chemotherapy.

In another preferred embodiment, the cancer is recurrent or metastatic cancer.

In another preferred embodiment, the cancer is cancer stem cell.

In another preferred embodiment, the cancer is cancer with stem cell properties.

In another preferred embodiment, the cancer is selected from the group consisting of cancer containing FGFR3-TACC3 fusion gene, cancer with high expression of BACH1 transcription factor, cancer with high expression of myc protein, cancer with kras mutation, cancer with glycolysis deficiency, cancer with TP53 gene mutation, cancer with BRAF gene mutation, cancer with CDKN2A gene mutation, cancer with PTEN gene mutation, cancer with CDKN2C gene mutation, cancer with CTNNB1 gene mutation, cancer with EGFR gene mutation, cancer with NRAS gene mutation, cancer with STK11 gene mutation, cancer with BARF gene mutation, cancer with SMAD4 gene mutation, cancer with MAP2K4 gene mutation, cancer with FBXW7 gene mutation, cancer with KDM6A gene mutation, cancer with BRCA2 gene mutation, cancer with BRCA1 gene mutation, cancer with RB1 gene mutation, cancer with CDH1 gene mutation, cancer with PIK3CA gene mutation, cancer with NPM1 gene mutation, cancer with DNMT3A R882C mutation, and combinations thereof.

In another preferred embodiment, the brain cancer is selected from the group consisting of glioma, medulloblastoma;

the breast cancer is selected from the group consisting of triple negative breast cancer, breast ductal adenocarcinoma, breast squamous cell carcinoma, metastatic breast cancer, and combinations thereof;

the liver cancer is anaplastic and lowly differentiated liver cancer;

the melanoma is selected from the group consisting of multidrug-resistant melanoma, malignant melanoma, and combinations thereof;

the leukemia is selected from the group consisting of myeloid leukemia, T lymphocyte leukemia, and combinations thereof;

the lung cancer is selected from the group consisting of small cell lung cancer, non-small cell cancer, and combinations thereof;

the lymphoma is selected from the group consisting of B cell lymphoma, mononuclear cell lymphoma, T cell lymphoma, and combinations thereof;

the pancreatic cancer is selected from the group consisting of pancreatic ductal adenocarcinoma, liver metastatic pancreatic cancer, and combinations thereof;

the kidney cancer is selected from the group consisting of kidney rhabdoid carcinoma, kidney smooth muscle carcinoma, kidney cell adenocarcinoma, and combinations thereof; and/or

the intestinal cancer is colorectal adenocarcinoma.

In another preferred embodiment, the brain cancer is selected from the group consisting of glioma and medulloblastoma.

In another preferred embodiment, the glioma comprises glioma with CDKN2A, PTEN and/or CDKN2C gene mutation.

In another preferred embodiment, the glioma is malignant glioma.

In another preferred embodiment, the glioma is glioblastoma.

In another preferred embodiment, the brain cancer is brain glial cell carcinoma.

In another preferred embodiment, the brain cancer is brain glioblastoma.

In another preferred embodiment, the glioblastoma is glioblastoma multiforme.

In another preferred embodiment, the brain cancer is medulloblastoma.

In another preferred embodiment, the medulloblastoma is cerebellar medulloblastoma.

In another preferred embodiment, the breast cancer is selected from the group consisting of triple negative breast cancer, breast ductal adenocarcinoma, breast squamous cell carcinoma, metastatic breast cancer, and combinations thereof.

In another preferred embodiment, the breast ductal adenocarcinoma is invasive breast ductal adenocarcinoma.

In another preferred embodiment, the invasive breast ductal adenocarcinoma comprises invasive breast ductal adenocarcinoma with PTEN, RB1 and/or TP53 gene mutation.

In another preferred embodiment, the breast squamous cell carcinoma is breast acantholysis squamous cell carcinoma.

In another preferred embodiment, the breast acantholysis squamous cell carcinoma is breast TNM IIB stage 2 primary acantholysis squamous cell carcinoma.

In another preferred embodiment, the breast acantholysis squamous cell carcinoma comprises breast acantholysis squamous cell carcinoma with CDKN2A, STK11, KDM6A and/or TP53 gene mutation.

In another preferred embodiment, the metastatic breast cancer comprises breast cancer with CDH1 and/or PIK3CA gene mutation.

In another preferred embodiment, the liver cancer is anaplastic and lowly differentiated liver cancer.

In another preferred embodiment, the liver cancer comprises liver cancer with CTNNB1 and/or NRAS gene mutation.

In another preferred embodiment, the liver cancer is grade II-III/IV liver cancer.

In another preferred embodiment, the melanoma is selected from the group consisting of multidrug-resistant melanoma, malignant melanoma, and combinations thereof.

In another preferred embodiment, the malignant melanoma comprises malignant melanoma with BRAF, CDKN2A and/or STK11 gene mutation.

In another preferred embodiment, the malignant melanoma is metastatic malignant melanoma.

In another preferred embodiment, the malignant melanoma is malignant melanoma with inguinal lymph node metastasis.

In another preferred embodiment, the leukemia is selected from the group consisting of myeloid leukemia, T lymphocyte leukemia and combinations thereof.

In another preferred embodiment, the myeloid leukemia is acute myeloid leukemia (AML).

In another preferred embodiment, the acute myeloid leukemia is M4 grade AML acute myeloid leukemia.

In another preferred embodiment, the acute myeloid leukemia is FAB M4 grade AML acute myeloid leukemia.

In another preferred embodiment, the FAB M4 grade acute myeloid leukemia comprises FAB M4 grade acute myeloid leukemia with NPM1 and/or R882C gene mutation.

In another preferred embodiment, the T lymphocyte leukemia is acute T lymphocyte leukemia.

In another preferred embodiment, the lung cancer is selected from the group consisting of small cell lung cancer, non-small cell lung cancer, and combinations thereof.

In another preferred embodiment, the lymphoma is selected from the group consisting of B cell lymphoma, mononuclear cell lymphoma, T cell lymphoma, and combinations thereof.

In another preferred embodiment, the lymphoma is Non-Hodgkin's lymphoma (NHL).

In another preferred embodiment, the T cell lymphoma is cutaneous T cell lymphoma.

In another preferred embodiment, the lymphoma is selected from the group consisting of B cell lymphoma, mononuclear cell lymphoma, cutaneous T cell lymphoma, and combinations thereof.

In another preferred embodiment, the pancreatic cancer is selected from the group consisting of pancreatic ductal adenocarcinoma, liver metastatic pancreatic cancer, and combinations thereof.

In another preferred embodiment, the pancreatic ductal adenocarcinoma comprises pancreatic ductal adenocarcinoma with TP53 gene mutation.

In another preferred embodiment, the kidney cancer is selected from the group consisting of kidney rhabdoid carcinoma, kidney smooth muscle carcinoma, kidney cell adenocarcinoma, and combinations thereof.

In another preferred embodiment, the kidney cell adenocarcinoma is metastatic kidney cell adenocarcinoma.

In another preferred embodiment, the kidney cell adenocarcinoma is primary kidney cell adenocarcinoma.

In another preferred embodiment, the intestinal cancer is colorectal adenocarcinoma.

In another preferred embodiment, the colorectal adenocarcinoma is selected from the group consisting of Dukes' type B colorectal adenocarcinoma, Dukes' type C, grade IV colorectal adenocarcinoma, and combinations thereof.

In another preferred embodiment, the Dukes' type C, grade IV colorectal adenocarcinoma comprises Dukes' type C, grade IV colorectal adenocarcinoma with CTNNB1, EGFR and/or FBXW7 gene mutation.

In another preferred embodiment, the colorectal cancer is colorectal adenocarcinoma.

In another preferred embodiment, the cancer is cancer with upregulation of the oxidative phosphorylation pathway of mitochondria and/or low activity of the mitochondria permeability transition pore in cancer cell.

In another preferred embodiment, the upregulation of the oxidative phosphorylation pathway of mitochondria means that the ratio (E1/E0) of the level or expression E1 of the oxidative phosphorylation pathway of mitochondria in a cell (such as cancer cell) to the level or expression E0 of the oxidative phosphorylation pathway of mitochondria in a normal cell (the same type of cell) is ≥1.2, preferably ≥1.5, more preferably ≥2, more preferably ≥3, more preferably ≥5.

In another preferred embodiment, the low activity of the mitochondria permeability transition pore means that the ratio (A1/A0) of the activity level or expression level A1 of the mitochondria permeability transition pore in a cell (such as cancer cell) to the activity level or expression level A0 of the mitochondria permeability transition pore in a normal cell (the same type of cell) is ≤0.8, preferably ≤0.7, more preferably ≤0.6, more preferably ≤0.5, more preferably ≤0.4, more preferably ≤0.3, more preferably ≤0.2, more preferably ≤0.1, most preferably ≤0.05.

In another preferred embodiment, the cancer patient is administered mitochondria permeability transition pore inhibitor to make the mitochondria permeability transition pore of cancer cell low activity.

In another preferred embodiment, the low activity of mitochondria permeability transition pore is made by administering the mitochondria permeability transition pore inhibitor.

In another preferred embodiment, the level is protein level and/or mRNA level.

In another preferred embodiment, the expression is protein expression and/or mRNA expression.

In another preferred embodiment, the mitochondria permeability transition pore inhibitor is selected from the group consisting of Cyclosporin A, CyP-D protein inhibitor, peroxide scavenger, and combinations thereof.

In another preferred embodiment, the virus is selected from the group consisting of influenza virus, parainfluenza virus, cytomegalovirus, adenovirus, rhinovirus, coronavirus, coxsackie virus, eko virus, chickenpox, rubella, measles virus, respiratory syncytial virus.

In another preferred embodiment, the virus is coronavirus.

In another preferred embodiment, the coronavirus is selected from the group consisting of α coronavirus, β coronavirus, and combinations thereof.

In another preferred embodiment, the coronavirus is selected from the group consisting of HCoV-229E, HCoV-OC43, SARS-CoV, HCoV-NL63, HCoV-HKU1, MERS-CoV, 2019-nCov, and combinations thereof.

In another preferred embodiment, the coronavirus is selected from the group consisting of 2019 novel Coronavirus (2019-nCov) SARS virus, MERS virus, and combinations thereof.

In another preferred embodiment, the coronavirus is selected from the group consisting of HCoV-229E, HCoV-OC43, SARS-CoV, HCoV-NL63, HCoV-HKU1, MERS-CoV, 2019-nCov, and combinations thereof.

In another preferred embodiment, the viral infection related disease is selected from the group consisting of pneumonia, pulmonary fibrosis, and combinations thereof.

In another preferred embodiment, the composition or preparation further comprises other anticancer drugs.

In another preferred embodiment, the other anticancer drugs are selected from the group consisting of immunotherapy drugs, chemotherapeutic drugs that block DNA synthesis, anticancer drugs that promote cell death, inhibitors targeting proteasome, and combinations thereof.

In another preferred embodiment, the immunotherapy drug is selected from the group consisting of PD1/PDL1, CTLA-4, and combinations thereof.

In another preferred embodiment, the chemotherapeutic drug that block DNA synthesis is selected from the group consisting of tegafur, fluorouracil, oxaliplatin, temozolomide, and combinations thereof.

In another preferred embodiment, the anticancer drugs that promote cell death is a Bcl-2 small molecule inhibitor (e.g., Venetoclax).

In another preferred embodiment, the inhibitor targeting proteasome is Bortezomib.

In another preferred embodiment, the composition or preparation further comprises a pharmaceutically acceptable carrier.

In another preferred embodiment, the composition is a pharmaceutical composition.

In another preferred embodiment, the pharmaceutical composition further comprises a pharmaceutically acceptable carrier.

In the second aspect of the present invention, it provides a compound of formula I or an optical isomer thereof, or a racemate thereof, or a solvate thereof, or a pharmaceutically acceptable salt thereof

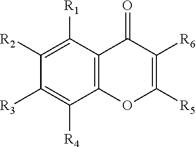

wherein,

R₁, R₂, R₃, R₄, R₅ and R₆ are each independently hydrogen, halogen, —CN, hydroxyl, sulfhydryl, nitro, amino, —COOH, —CHO, substituted or unsubstituted C1-C12 alkyl, substituted or unsubstituted C3-C16 cycloalkyl, substituted or unsubstituted C1-C12 alkoxyl, substituted or unsubstituted C1-C12 alkylthio, substituted or unsubstituted 3-16 membered heterocycloalkyl, substituted or unsubstituted C6-C16 aryl, substituted or unsubstituted 3-16 membered heteroaryl, substituted or unsubstituted glycosyl-O—, substituted or unsubstituted glycosyl-S—, substituted or unsubstituted uronic acid group-O—, or substituted or unsubstituted uronic acid group-S—;

each “substituted” means that one or more (preferably 1, 2, 3, or 4) hydrogen atoms on the group are substituted by a substituent selected from the group consisting of C1-C8 alkyl, C3-C8 cycloalkyl, C1-C8 haloalkyl, C3-C8 halocycloalkyl, halogen, nitro, —CN, hydroxyl, sulfhydryl, amino, C1-C4 carboxyl, C2-C4 ester group, C2-C4 amido, C1-C8 alkoxyl, C1-C8 alkylthio, C1-C8 haloalkoxyl, C1-C8 haloalkylthio, C6-C12 aryl, 5-10 membered heteroaryl, 5-10 membered heterocycloalkyl;

the heterocyclic ring of the heterocycloalkyl and heteroaryl each independently contains 1-4 (preferably 1, 2, 3 or 4) heteroatoms selected from the group consisting of N, O and S.

In another preferred embodiment, R₁, R₂, R₃, R₄, R₅ and R₆ are each independently as described in the first aspect of the present invention.

In another preferred embodiment, the compound of formula I is as described in the first aspect of the present invention.

In the third aspect of the present invention, it provides a pharmaceutical composition comprising (a) the compound of formula I, or an optical isomer thereof, or racemate thereof, or a solvate thereof, or a pharmaceutically acceptable salt thereof according to the second aspect of the present invention; and (b) a pharmaceutically acceptable carrier.

In another preferred embodiment, the pharmaceutical composition further comprises other anticancer drugs.

In another preferred embodiment, the other anticancer drugs are as described in the first aspect of the present invention.

In the fourth aspect of the present invention, it provides a use of a mitochondria permeability transition pore inhibitor for preparing a composition or preparation, and the composition and preparation are used to enhance the anticancer effect of anticancer drug.

In another preferred embodiment, the anticancer drug is the compound of formula I, or an optical isomer thereof, or a racemate thereof, or a solvate thereof, or a pharmaceutically acceptable salt thereof according to the first aspect of the present invention, and/or other anticancer drugs.

In another preferred embodiment, the cancer is as described in the first aspect of the present invention.

In another preferred embodiment, the mitochondria permeability transition pore inhibitor is selected from the group consisting of Cyclosporin A, CyP-D protein inhibitor, peroxide scavenger, and combinations thereof.

In another preferred embodiment, the CyP-D protein inhibitor is SfA, BKA and ADP (small molecule that regulate the activity of ANT protein).

In another preferred embodiment, the peroxide scavenger is selected from the group consisting of propofol, pyruvate, MCI-186, and combinations thereof.

In the fifth aspect of the present invention, it provides an active ingredient combination, the active ingredient combination comprise:

(1) a first active ingredient, the first active ingredient is anticancer drug; and

(2) a second active ingredient, the second active ingredient is mitochondria permeability transition pore inhibitor.

In another preferred embodiment, the molar ratio of the first active ingredient to the second active ingredient is 0.01-600:1, preferably 0.05-500:1, more preferably 0.1-400:1, more preferably 0.2-200:1, more preferably 0.5-100:1, more preferably 0.5-80:1, most preferably 1-50:1.

In another preferred embodiment, at least one active ingredient is independent in the active ingredient combination.

In another preferred embodiment, the first active ingredient and the second active ingredient are independent of each other in the active ingredient combination.

In another preferred embodiment, the active ingredient combination further comprises other anticancer drugs.

In the sixth aspect of the present invention, it provides a composition comprising:

(1) a first active ingredient, the first active ingredient is anticancer drug; and

(2) a second active ingredient, the second active ingredient is mitochondria permeability transition pore inhibitor.

In another preferred embodiment, the composition is a pharmaceutical composition.

In another preferred embodiment, the pharmaceutical composition further comprises a pharmaceutically acceptable carrier.

In another preferred embodiment, the content of the fist active ingredient is 0.01-99.99 wt %, preferably 0.1-99.9 wt %, more preferably 1-99 wt %, more preferably 10-99 wt %, most preferably 20-99 wt %, based on the total weight of the active ingredient of the composition.

In another preferred embodiment, the content of the second active ingredient is 0.01-99.99 wt %, preferably 0.1-99.9 wt %, more preferably 1-99 wt %, more preferably 10-99 wt %, and most preferably 20-99 wt %, based on the total weight of the active ingredients of the composition.

In the seventh aspect of the present invention, it provides a medical kit, the medical kit comprises:

(A) a first preparation comprising a first active ingredient, the first active ingredient is anticancer drug; and

(B) a second preparation comprising a second active ingredient, the second active ingredient is mitochondria permeability transition pore inhibitor.

In another preferred embodiment, the medical kit further comprises user's manual.

In another preferred embodiment, the first preparation and the second preparation are independent of each other.

In another preferred embodiment, the first preparation and the second preparation are combined preparation.

In another preferred embodiment, the users manual records that the first preparation and the second preparation are used in combination to enhance the anti-tumor activity of the anticancer drug.

In another preferred embodiment, the method for use in combination is to administer the second preparation comprising the mitochondria permeability transition pore inhibitor first, and then administer the anticancer drug.

In another preferred embodiment, the medical kit further comprises other anticancer drugs.

In an eighth aspect of the present invention, it provides a method for non-therapeutically and non-diagnostically inhibiting oxidative phosphorylation pathway of mitochondria in vitro, which comprises contacting the oxidative phosphorylation pathway of mitochondria or cell expressing the oxidative phosphorylation pathway of mitochondria with the compound of formula I, or an optical isomer thereof, or a racemate thereof, or a solvate thereof, or a pharmaceutically acceptable salt thereof according to the second aspect of the invention, thereby inhibiting oxidative phosphorylation pathway of mitochondria.

In the ninth aspect of the present invention, it provides a method for non-therapeutically and non-diagnostically inhibiting cancer cell in vitro, which comprises contacting the cancer cell with the compound of formula I, or an optical isomer thereof, or racemate thereof, or a solvate thereof, or a pharmaceutically acceptable salt thereof according to the first or second aspect of the invention, thereby inhibiting cancer cell.

In another preferred embodiment, the contact is performed in vitro culture.

In the tenth aspect of the present invention, it provides a method for (a) inhibiting the oxidative phosphorylation pathway of mitochondria; (b) preventing and/or treating diseases associated with oxidative phosphorylation pathway of mitochondria; and/or (c) preventing and/or treating cancers, which comprises administering the compound of formula I, or an optical isomer thereof, or a racemate thereof, or a solvate thereof, or a pharmaceutically acceptable salt thereof according to the first or second aspect of the present invention, or the active ingredient combination according to the fifth aspect of the present invention, or the composition according to the sixth aspect of the present invention, or the medical kit according to the seventh aspect of the present invention to a subject in need, thereby (a) inhibiting the oxidative phosphorylation pathway of mitochondria; (b) preventing and/or treating diseases associated with oxidative phosphorylation pathway of mitochondria; and/or (c) preventing and/or treating cancers.

In another preferred embodiment, the subject is human and non-human mammals (rodent, rabbit, monkey, livestock, dog, cat, and the like).

It should be understood that, in the present invention, each of the technical features specifically described above and below (such as those in the Examples) can be combined with each other, thereby constituting new or preferred technical solutions which need not be redundantly described one-by-one.

### DESCRIPTION OF THE DRAWINGS

FIG. 1 shows the inhibitory effect of Compound SJ2858 (baicalein) and Compound SJ2775 (baicalin) on oxidative phosphorylation pathway of mitochondria (repeated three times in parallel).

### EMBODIMENTS FOR CARRYING OUT THE INVENTION

Based on an extensive and intensive research, the inventors have unexpectedly found a specific class of scutellariae radix compounds (the compound of formula I, or an optical isomer thereof, or a racemate thereof, or a solvate thereof, or a pharmaceutically acceptable salt thereof) can safely and effectively inhibit the oxidative phosphorylation pathway of mitochondria. The experimental results showed that the compound of the present invention can significantly inhibit the activity of tumor cells with unregulated oxidative phosphorylation pathway of mitochondria (or inactive mPTP) at a low concentration (IC50), indicating the compound of the present invention has significant inhibitory effect on tumor cells with lowly active or inactive mPTP, while the inhibitory effect of the compound of the present invention on normal cells with highly active mPTP is weak, and the compound of the present invention has little toxic and side effects. On this basis, the inventors has completed the present invention.

Terms

As used herein, the term “comprise”, “comprising”, and “containing” are used interchangeably, which not only comprise closed definitions but also semi-closed and open definitions. In other words, the term comprises “consisting of” and “essentially consisting of”.

The term “anticancer drug” and “anti-tumor drug” are used interchangeably.

The term “cancer” and “tumor” are used interchangeably.

The term “IC50” is 50% inhibiting concentration, ie, the concentration of the inhibitor when 50% inhibitory effect is achieved.

The term “mitochondria permeability transition pore” is abbreviated as mPTP.

The term “Oxidative Phosphorylation Pathway” is abbreviated as OXPHOS (Oxidative Phosphorylation), also known as oxidative phosphorylation.

The term “cancer stem cell”, also known as tumor stem cell, tumor cell with strong stem cell properties tumor cell with low differentiation, or slow circulating tumor cells, etc, refers to those with stem cell properties and the ability of self-renewal and multi-cellular differentiation, etc. Cancer stem cell has strong ability to form tumor, especially after the cancer metastasizes, it can generate new types of cancer. Clinically, cancer stem cell are mainly tumor cell that are not sensitive to radiotherapy and chemotherapy, radiotherapy and chemotherapy have no killing effect on them. After radiotherapy and chemotherapy, cancer stem cell can rapidly divide and proliferate which is an important reason for the recurrence of various malignant tumors.

It should be understood that the skilled in the art can choose the substituents and substituted forms on the compound of the present invention to obtain chemically stable compounds, the compound can be synthesized by the techniques known in the art and the methods described below. If the compound is substituted by more than one substituents, it should be understood that the substituents can be on the same carbon or on different carbons, as long as a stable structure is obtained.

As used herein, the term “substitute” or “substituted” means the hydrogen atom on the group is substituted by a non-hydrogen atom group but it needs to meet its valence requirements and the substituted compound is chemically stable, that is, the substituted compound does not spontaneously undergo transformations such as cyclization and elimination, etc.

As used herein, “R₁”, “R1” and “R₁” have the same meaning and can be used interchangeably. The other similar definitions have the same meaning.

As used herein, “ ” denotes the linking site of the group.

As used herein, the term “alkyl” refers to a linear chain (ie, unbranched) or branched saturated hydrocarbon group containing only carbon atoms or a combination of linear and branched chains. When the number of carbon atoms is limited in front of the alkyl (eg C1-C10 alkyl), it means that the alkyl has 1-10 carbon atoms, for example, C1-C4 alkyl refers to an alkyl having 1-4 carbon atoms. Representative examples comprise but are not limited to methyl, ethyl, propyl, isopropyl, butyl, isobutyl, sec-butyl, tert-butyl, or the like.

As used herein, the term “halogen” refers to F, Br or I.

As used herein, the term “halo” means the group is substituted by halogen.

As used herein, the term “haloalkyl” means that one or more (preferably 1, 2, 3 or 4) hydrogens on alkyl are substituted by halogen, the alkyl and halogen are as defined above. When the number of carbon atoms in front of the alkyl is limited (e.g., C1-C6 haloalkyl), it means that the alkyl has 1-6 carbon atoms, for example, C1-C6 haloalkyl refers to an haloalkyl having 1-6 carbon atoms. Representative examples comprise but are not limited to —CF3, —CHF2, monofluoroisopropyl, difluorobutyl, or the like.

As used herein, the term “cycloalkyl” refers to a cyclic group having a saturated or partially saturated monocyclic ring, bicyclic ring or polycyclic ring (fused ring, bridged ring or spiro ring) system. When the number of carbon atoms is limited in front of the cycloalkyl (e.g., C3-C12 cycloalkyl), it means the cycloalkyl has 3-12 ring carbon atoms. In some preferred embodiments, C3-C8 cycloalkyl refers to a saturated or partially saturated monocyclicalkyl or bicyclic alkyl having 3-8 ring carbon atoms, comprising cyclopropyl, cyclobutyl, cyclopentane, cycloheptyl, or the like. “Spirocycloalkyl” refers to a bicyclic or polycyclic group in which a single carbon atom (called spiro atom) is shared by monocyclic rings, spirocycloalkyl can have one or more double bonds, but none of the rings has completely conjugated π electron system. “Fused cycloalkyl” refers to an all-carbon bicyclic or polycyclic group in which each ring shares an pair of adjacent carbon atoms with other rings in the system, wherein one or more rings can have one or more double bonds, but none of the rings has completely conjugated π electron system. “Bridged cycloalkyl” refers to an all-carbon polycyclic group in which any two rings share two carbon atoms that are non-directly attached, the bridged cycloalkyl rings can have one or more double bonds, but none of the rings have completely conjugated π electron system. Representative examples of cycloalkyl are but not limited to, as follows:

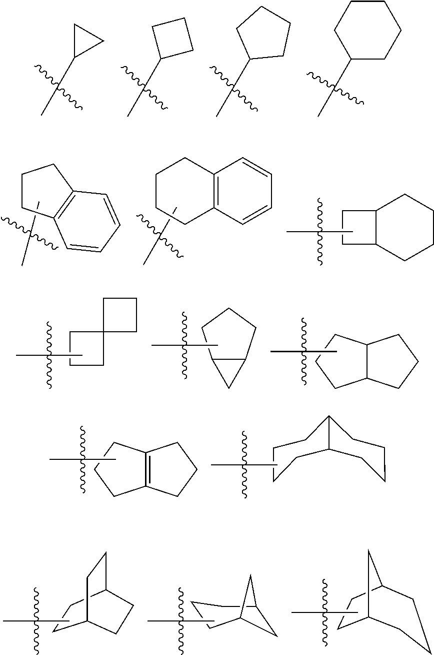

As used herein, the term “halocycloalkyl” means that one or more (preferably 1, 2, 3 or 4) hydrogens on cycloalkyl are substituted by halogen, the cycloalkyl and halogen are as defined above. When the number of carbon atoms is limited in front of the cycloalkyl (e.g., C3-C8 haloalkyl), it means that the cycloalkyl has 3-8 ring carbon atoms, for example, C3-C8 haloalkyl refers to an halocycloalkyl having 3-6 carbon atoms. Representative examples comprises but are not limited to monofluorocyclopropyl, monochlorocyclobutyl, monofluorocyclopentyl, difluorocycloheptyl or the like.

As used herein, the term “alkoxyl” refers to R—O— group, wherein R is alkyl, and the alkyl is as defined above. When the number of carbon atoms is limited in front of the alkoxyl, for example, C1-C8 alkoxyl means that the alkyl in the alkoxyl has 1-8 carbon atoms. Representative examples of alkoxyl comprise but are not limited to methoxyl, ethoxyl, n-propoxyl, isopropoxyl, tert-butoxyl, or the like.

As used herein, the term “alkylthio” refers to R—O— group, wherein R is alkyl, and the alkyl is as defined above. When the number of carbon atoms is limited in front of the alkylthio, for example, C1-C8 alkylthio means that the alkyl in the alkylthio has 1-8 carbon atoms. Representative examples of alkylthio comprise but are not limited to methylthio, ethylthio, n-propylthio, isopropylthio, tert-butylthio, or the like.

As used herein, the term “haloalkoxyl” refers to haloalkyl-O—, wherein the haloalkyl is as defined above, for example, C1-C6 haloalkoxyl refers to a haloalkoxyl having 1-6 carbon atoms. Representative examples of alkylthio comprise but are not limited to monofluoromethoxyl, monofluoroethoxyl, bisfluorobutoxyl, or the like.

As used herein, the term “haloalkylthio” refers to haloalkyl-S—, wherein the haloalkyl is as defined above, for example, C1-C6 haloalkylthio refers to a haloalkylthio having 1-4 carbon atoms. Representative examples of alkylthio comprise but are not limited to monofluoromethylthio, monofluoroethylthio, difluorobutylthio, or the like.

The term “heterocycloalkyl” refers to fully saturated or partially unsaturated cyclic group (comprising but is not muted to such as 3-7 membered monocyclic ring, 7-11 membered bicyclic ring, or 8-16 membered tricyclic ring), at least one heteroatom is present in a ring with at least one carbon atom. When the number of members is limited in front of the heterocycloalkyl, it refers to the number of ring atoms of the heterocycloalkyl, for example, 3-16 membered heterocycloalkyl refers to a heterocycloalkyl having 3-16 ring atoms. Each heterocyclic ring having heteroatoms can have one or more (e.g., 1, 2, 3 or 4) heteroatoms, each of heteroatoms is independently selected from the group consisting of nitrogen atom, oxygen atom or sulfur atom, wherein nitrogen atom or the sulfur atom can be oxidized, and the nitrogen atom can also be quaternized. Heterocycloalkyl can be attached to any heteroatom or carbon atom residue of ring or ring system molecule. Representative examples of monocyclic heterocycloalkyl comprise but are not limited to azetidinyl, oxetanyl imidazolinyl, imidazolidinyl, tetrahydrofuranyl, piperidinyl, piperazinyl, 2-oxopiperazinyl, 2-oxopiperidinyl, 4-piperidone group, tetrahydropyranyl, morpholinyl, thiomorpholinyl, thiomorpholinyl sulfoxide, thiomorpholinyl sulfonyl, 1,3-dioxanyl and tetrahydro-1,1-dioxythiophene, etc. Polycyclic heterocycloalkyl comprises heterocyclyl with spiro ring, fused ring and bridged ring, the heterocycloalkyl with the spiro ring, fused ring and bridge ring are optionally linked with other groups by single bond, or further linked with other cycloalkyl rings and heterocyclic rings by any two or more atoms on the ring.

The term “aryl” refers to an all carbon monocyclic ring or fused polycyclic ring (i.e., a ring that share adjacent carbon atom pairs) groups with a conjugated π electron system, which is aromatic cyclic hydrocarbon compound group. When the number of carbon atoms is limited in front of the aryl, for example, C6-C12 aryl means that the aryl has 6-12 ring carbon atoms, such a phenyl and naphthyl. The aryl can be fused to other cyclic groups (comprising saturated or unsaturated rings), but cannot have heteroatoms such as nitrogen, oxygen, or sulfur, and the site linking to the parent must be on the carbon atom of the ring with a conjugated π electron system. Representative examples of aryl are, but not limited to, as follows:

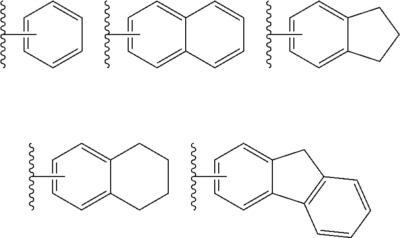

The term “heteroaryl” refers to aromatic heterocyclic ring group having one to more (preferably 1, 2, 3 or 4) heteroatoms, the heteroaryl can be monocyclic ring (monocyclic), or polycyclic ring (bicyclic, tricyclic or polycyclic) fused together or covalently connected. Each of heterocyclic ring having heteroatom can have one or more (e.g., 1, 2, 3 4) heteroatoms independently selected from the group consisting of oxygen, sulfur and nitrogen. When the number of members is limited in front of the heteroaryl, it refers to the number of ring atoms of the heteroaryl, for example, 5-12 membered heteroaryl refers to a heteroaryl having 5-12 ring atoms. Representative examples comprise but are not limited to pyrrolyl, pyrazolyl, imidazolyl, oxazolyl, isoxazolyl, thiazolyl, thiadiazolyl, isothiazolyl, furanyl, pyridyl, pyrazinyl, pyrimidinyl, pyridazinyl, triazinyl, triazolyl and tetrazolyl, etc.

As used herein, the term “carboxyl” refers to —COOH or -alkyl-COOH, the alkyl is as defined above, for example, C2-C4 carboxyl refers to —C1-C3 alkyl-COOH. Representative examples comprise but are not limited to —COOH, —CH₂COOH, —C₂H₄COOH, or the like.

As used herein, the term “ester group” refers to R—CO—O— or —CO—O—R, wherein R is alkyl, the alkyl is as defined above, for example, C₂-C₄ ester group refers to C₁-C₃ alkyl-CO—O— or —CO—O—C₁-C₃ alkyl. Representative examples comprise but are not limited to CH₃COO—, C₂H₅COO—, C₃H₈COO—, (CH₃)₂CHCOO—, —COOCH₃, —COOC₂H₅, —COOC₃H₈, or the like.

As used herein, the term “amido” refers to R—CO—N— or —CO—N—R, wherein R is alkyl, the alkyl is as defined above, for example, C2-C4 amido refers to C₁-C₃ alkyl-CO—N— or —CO—N—C₁-C₃ alkyl. Representative examples comprise but are not limited to CH₃CO—N—, C₂H₅CO—N—, C₃H₈CO—N—, (CH₃)₂CHCO—N—, —CO—N—CH₃, —CO—N—C₂H₅, —CO—N—C₃H₈, or the like.

As used herein, alone or as part of other substituents, the term “amino” is —NH₂.

As used herein, alone or as part of other substituents, the term “nitro” is —NO₂.

As used herein, alone or as part of other substituents, the term “cyano” is —CN.

As used herein, alone or as part of other substituents, the term “hydroxyl” is —OH.

As used herein, alone or as part of other substituents, the term “sulfhydryl ” is —SH.

As used herein, the term “glycose” is a polyhydroxy (two or more) aldehyde or ketone compound. Chemically, the glycose is composed of carbon, hydrogen and oxygen elements and similar to the polymerization of “carbon” and “water” in the chemical property. Representatively, the glycose is monoglycose, bisglycose, oligoglycose or polyglycose.

As used herein, the term “monoglycose” refers to a glycose that cannot be further hydrolyzed, and is the basic molecule unit forming various bisglycoses and polyglycoses. The preferred monoglycose refers to a monoglycose containing 3-6 carbon atoms in the molecular structure, such as glyceraldehyde of three-carbon glycose (triose); erythrose and threose of four-carbon glycose (butanose); arabinose, ribose, xylose, lyxose of five-carbon glycose (pentose); glucose, mannose, fructose, galactose of the six-carbon glycose (hexose). According to the number of carbon atom, monoglycose can be classified into triose, butanose, pentose, hexose, etc. According to the structure, monoglycose can be further classified into aldose and ketose. Polyhydroxy aldehyde can be called aldose and polyhydroxy ketone can be called ketose. For example, glucose is aldohexose and fructose is ketohexose. The hydroxyl in the monoglycose molecule can be reversibly condensed with the aldehyde group or the ketone group to form cyclic emiacetal. After cyclization, carbonyl C transforms into chiral C atom called anomeric carbon atom, and the two diastereomers formed after cyclization are called terminal isomers or head isomers. It should be understood that the monoglycose of the present invention comprises open-chain structure, cyclic hemiacetal formed inside the molecule, and a mixture thereof.

As used herein, the term “bisglycose” is formed from composed of two monoglycose through glycosidic bond. In the case that the reducing group of one monoglycose is linked with the alcoholic hydroxyl of the other glycose, it shows similar chemical properties to monoglycoses, such as the reduction in Fehling solution, mutarotation, osazone formation (such as maltose and lactose), monoglycoses (e.g., sucrose, trehalose, lactose) linked by reducing groups can no have this property.

As used herein, the term “oligoglycose” refers to a glycose chain composed of 3-9 monoglycoses linked by glycosidic bonds. Oligoglycoses composed of the same monoglycose are called homo-oligoglycoses, and oligoglycoses composed of different monoglycoses are called hetero-oligoglycoses. Representatively, the oligoglycose is cyclodextrin or similar oligoglycose.

As used herein, the term “polyglycose” refers to a glycose chain linked by glycosidic bond and a polyglycose macromolecular carbohydrate composed of at least more than 10 monoglycoses. Polyglycoses composed of the same monoglycoses are called homopolyglycoses, such as starch, cellulose and glycogen; polyglycoses composed of different monoglycoses are called heteropolyglycoses, such as arabic gum composed of pentose and galactose. Polyglycose is not a pure chemical substance, but a mixture of substances with different degrees of polymerization. Polyglycoses are generally insoluble in water, have no sweet taste, cannot form crystals, and have no reducibility and mutarotation. Polyglycoses are also glycosides, so they can be hydrolyzed. In the process of the hydrolysis, a series of intermediate products are often produced, and finally polyglycoses are completely hydrolyzed to produce monoglycoses. Representatively, the polyglycose is starch, glycogen, cellulose, chitin, inulin or agar, or similar polyglycoses.

The term “glycosyl” refers to a monovalent substituent formed by removing hemiacetal hydroxyl from glycose such as monoglycose, bisglycose, oligoglycose or polyglycoses. Hemiacetal hydroxyl means that hydrogen atoms on some hydroxyl in glycoses such as polyhydroxyaldehyde or polyhydroxyketone can spontaneously undergo addition reaction with ketone group (carbonyl group), thus generating a hydroxyl, which is called hemiacetal hydroxyl. Hemiacetal hydroxyl is a kind of hydroxyl ether structure located in the ring structure.

As used herein, the term “uronic acid group” refers to a group formed by the oxidation of one or more (preferably 1, 2 or 3) primary hydroxyl groups in glycosyl to carboxyl, and the glycosyl is as defined above. The uronic acid group is easy to form lactone, and the uronic acid group is in equilibrium with its corresponding lactone. It should be understood that the uronic acid group described in the present invention comprises the equilibrium state of its lactone.

In present invention, it should be understood that all substituents are unsubstituted, unless explicitly described herein as “substituted”. The term “substituted” means that one or more hydrogen atoms on the specified group are substituted by specified substituent. The specific substituent is the substituent described above, or the substituent in each example. Preferably, the “substituted” means that one or more (preferably 1, 2, 3, or 4) hydrogen atoms on the group are substituted by a substituent selected from the group consisting of C1-C8 alkyl, C3-C8 cycloalkyl, C1-C8 haloalkyl, C3-C8 halocycloalkyl, halogen, nitro, —CN, hydroxyl, sulfhydryl, amino, C1-C4 carboxyl, C2-C4 ester group, C2-C4 amido, C1-C8 alkoxyl, C1-C8 alkylthio, C1-C8 haloalkoxyl, C1-C8 haloalkylthio, C6-C12 aryl, 5-10 membered heteroaryl, 5-10 membered heterocycloalkyl. Unless otherwise specified, each substituted group can have a substituent selected from a specified group at any substituted position of the group, the substitution can be the same or different at each substituted position.

In the present invention, the term “prevention” refers to a method of preventing the occurrence of disease and/or its accompanying symptoms, or protecting a subject from getting disease. “Prevention” as used herein also comprises delaying the occurrence of the disease and/or its accompanying symptoms and reducing the risk of the disease in a subject.

In the present invention, the term treatment comprises delaying and terminating the progression of the disease, or eliminating the disease, and does not require 100% inhibition, elimination and reversal. In some embodiments, compared to the level observed in the absence of the composition, medical kit, food kit or health care kit or active ingredient combination of the present invention, the composition or pharmaceutical composition of the present invention alleviates, inhibits and/or reverses related diseases (such as tumor) and its accompanying symptoms, eg, by at least about 10%, at least about 30%, at least about 50%, or at least about 80% by inhibiting the oxidative phosphorylation pathway of mitochondria.

Oxidative Phosphorylation Pathway of Mitochondria and Mitochondria Permeability Transition Pore

Oxidative Phosphorylation (OXPHOS) is one of the most important pathways in mitochondria, which utilizes NADH and FADH derived from tricarboxylic acid cycle and fat oxidation, etc to produce ATP. The oxidative phosphorylation pathway of mitochondria is composed of more than 90 proteins, which form five protein complexes, complexes I, II, III, IV and V. Studies have shown that the oxidative phosphorylation pathway of mitochondria is very important for cell growth and is related to many diseases such as cancers, immune-related diseases, neurodegenerative diseases, and viral infections. Inhibiting the oxidative phosphorylation pathway of mitochondria can treat cancers, immune related diseases, neurodegenerative diseases and viral infections, especially cancer cells with high malignancy and stem cell properties are extremely dependent on this pathway for survival. Inhibiting this pathway can effectively kill such cancer cells, thereby solving the problem of related malignant cancer recurrence.

Oxidative phosphorylation pathway of mitochondria is regulated by mitochondria permeability transition pore (mPTP). The compounds of the present invention have better inhibitory effect on oxidative phosphorylation pathway of mitochondria in the case that mPTP is inactive.

The study of the present invention has shown that the compound of the present invention has more significant inhibitory effect on cells with upregulated (or positive) oxidative phosphorylation pathway of mitochondria.

As used herein, the term “the upregulation of the oxidative phosphorylation pathway of mitochondria” and “positive oxidative phosphorylation pathway mitochondria” are used interchangeably and means that the level or expression of the oxidative phosphorylation pathway of mitochondria in a cell (such as cancer cell) is higher that the level or expression of the oxidative phosphorylation pathway of mitochondria in a normal cell (the same type of cell). Preferably, the term “the upregulation of the oxidative phosphorylation pathway of mitochondria” or “positive oxidative phosphorylation pathway mitochondria” means that the ratio (E1/E0) of the level or expression E1 of the oxidative phosphorylation pathway of mitochondria in a cell (such as cancer cell) to the level or expression E0 of the oxidative phosphorylation pathway of mitochondria in a normal cell (the same type of cell) is ≥1.2, preferably ≥1.5, more preferably ≥2, more preferably ≥3, more preferably ≥5.

In the present invention, “the upregulation of the oxidative phosphorylation pathway of mitochondria” and “the positive oxidative phosphorylation pathway mitochondria” can also be characterized by the activity of mPTP, and inactive mPTP means “the upregulation of the oxidative phosphorylation pathway of mitochondria” or “the positive oxidative phosphorylation pathway mitochondria”, for example, in the case that the ratio (A1/A0) of the activity level or expression level A1 of the mPTP in a cell (such as cancer cell) to the activity level or expression level A0 of the mPTP in a normal cell (the same type of cell) is ≤0.8, preferably ≤0.7, more preferably ≤0.6, more preferably ≤0.5, more preferably ≤0.4, more preferably ≤0.3, more preferably ≤0.2, more preferably ≤0.1, most preferably ≤0.05, it can be considered that the cell is upregulation of the oxidative phosphorylation pathway of mitochondria or positive oxidative phosphorylation pathway of mitochondria.

In a preferred embodiment, the level is protein level and/or mRNA level.

In a preferred embodiment, the expression is protein expression and/or mRNA expression.

In the present invention, the level or expression of oxidative phosphorylation pathway, the activity level or expression level of mPTP can be measured by conventional methods, such as measuring the activity of mPTP, or measuring the expression level of mPTP at the protein level or mRNA level.

Compound of Formula I

As used herein, the terms “compound of the present invention”, “compound of formula I of the present invention” and “compound of formula I” are used interchangeably, and refer to a compound of formula I, or an optical isomer thereof, or a racemate thereof, or a solvate thereof, or a pharmaceutically acceptable salt thereof. It should be understood that the term also comprises a mixture of the above components.

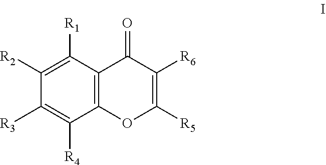

Specifically, the compound of formula I is as described above in the first aspect of the present invention.

The research of the present invention has showed that the compound of the present invention has inhibitory effect on the oxidative phosphorylation pathway of mitochondria, thus the compound of the present invention can be used as an active ingredient for inhibiting the oxidative phosphorylation pathway of mitochondria.

The compound of the present invention can treat diseases associated with the oxidative phosphorylation pathway of mitochondria by inhibiting the oxidative phosphorylation pathway of mitochondria, such as cancers, immune-related diseases, neurodegenerative diseases, viral infections and/or its related diseases, etc. Representatively, the compound of the present invention has significant inhibitory effect on tumor cell, and has more significant inhibitory effect on tumor cells with upregulated oxidative phosphorylation pathway of mitochondria or low mPTP activity especially on tumor cell with always closed mPTP. Studies have shown that mPTP has low activity in tumor cells with low differentiation. Therefore, the compound of the present invention can have a significantly therapeutic effect on tumor with low differentiation. At the same time, due to the high mPTP activity in normal somatic cells the compound of the present invention has weaker inhibition on normal somatic cells, and has less toxic and side effects, so the compound of the present invention can be developed into a relatively safe and effective anticancer drug.

The term “pharmaceutically acceptable salt” refers to a salt formed by a compound of the present invention and an acid or a base suitable for use as a medicine. Pharmaceutically acceptable salts comprises inorganic salts and organic salts. A preferred type of salt is the salt formed by the compound of the present invention and an acid. Acids suitable for salt formation comprise but are not limited to inorganic acid such as hydrochloric acid, hydrobromic acid, hydrofluoric acid, sulfuric acid, nitric acid, phosphoric acid and the like; organic acid such as formic acid, acetic acid, propionic acid, oxalic acid, malonic acid, succinic acid, fumaric acid, maleic acid, lactic acid, malic acid, tartaric acid, citric acid, picric acid, methanesulfonic acid, toluenesulfonic acid, benzenesulfonic acid and the like; and acidic amino acid such its aspartic acid and glutamic acid. A preferred type of salt is a metal salt formed by the compound of the present invention and a base. Suitable bases for salt formation comprise but are not limited to inorganic base such as sodium hydroxide, potassium hydroxide, sodium carbonate, sodium bicarbonate, sodium phosphate and the like; and organic base such as ammonia, triethylamine, diethylamine and the like.

The preferred compounds of the present invention is selected from the following group:

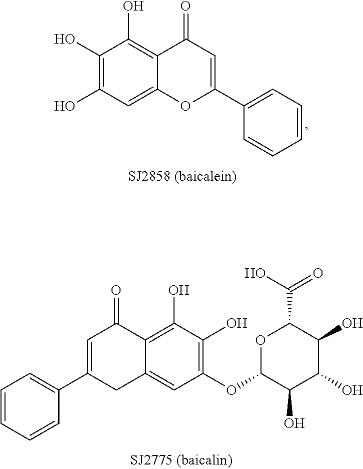 [SMILES: O=c1cc(-c2ccccc2)oc2cc(O)c(O)c(O)c12 ; O=C1C=C(c2ccccc2)Cc2cc(O[C@@H]3O[C@H](C(=O)O)[C@@H](O)[C@H](O)[C@H]3O)c(O)c(O)c21]

The compound of formula I in present invention can be converted into its pharmaceutically acceptable salt by conventional methods. For example, a solution of corresponding acid can be added into the solution of above compounds, and the solvent is removed after the salt is formed, thereby forming the corresponding salt of the compound of the present invention.

Diseases Associated with Oxidative Phosphorylation Pathway of Mitochondria

The compound of the present invention can be used to treat diseases associated with oxidative phosphorylation pathway of mitochondria, such as cancers, immune-related diseases, neurodegenerative disease, viral infection and/or its related diseases. Preferably, the diseases associated with oxidative phosphorylation pathway of mitochondria are diseases associated with the upregulation of the oxidative phosphorylation pathway of mitochondria or low activity of mPTP.

Representatively, the disease associated with the oxidative phosphorylation pathway of mitochondria are cancers.

Cancer

The studies of the present invention have shown that the compound of the present invention has significant inhibitory effect on tumor cell, especially on tumor cell with upregulation of the oxidative phosphorylation pathway of mitochondria or low activity of mPTP. The mPTP activity is extremely low in tumor cells with stem cell properties or low differentiation, so the compound of the present invention has more significant inhibitory effects on such cells.

In a preferred embodiment of the present invention, the cancer comprises but is not limited to lung cancer, pancreatic cancer, breast cancer, lymphoma, prostate cancer, brain cancer, leukemia, liver cancer, melanoma, intestinal cancer, kidney cancer, and combinations thereof.

In another preferred embodiment, the cancer comprises but is not limited to adenocarcinoma, ductal carcinoma, squamous cell carcinoma, and combinations thereof.

In another preferred embodiment, the cancer is lowly differentiated, moderately differentiated or highly differentiated cancer cell.

Preferably, the lowly differentiated cancer cell means that the ratio (L1/L2) of the differentiation degree L1 of the cancer cell to the differentiation degree L2 of normal tissue cell is ≤0.8, preferably ≤0.7, more preferably ≤0.6, more preferably ≤0.5, more preferably ≤0.4, more preferably ≤0.3, more preferably ≤0.2, more preferably ≤0.1, most preferably ≤0.05.

Preferably, the moderately differentiated cancer cell means that the ratio (M1/M2) of the differentiation degree M1 of the cancer cell to the differentiation degree M2 of normal tissue cell is 0.2-0.8, preferably 0.3-0.7, more preferably 0.4-0.6, most preferably 0.45-0.55.

Preferably, the highly differentiated cancer cell means the ratio (H1/H2) of the differentiation degree H1 of cancer cell to the differentiation degree H2 of normal tissue cell is 0.7-1.3, preferably 0.8-1.2, more preferably 0.9-1, most preferably 0.95-1.05.

In another preferred embodiment, the cancer is less differentiated cancer.

Preferably, the less differentiated cancer means that the ratio (D1/D2) of differentiation degree D1 of the cancer cell to the differentiation degree D2 of normal tissue cell is ≤0.8, preferably ≤0.7, more preferably ≤0.6, more preferably ≤0.5, more preferably ≤0.4, more preferably ≤0.3, more preferably ≤0.2, more preferably ≤0.1, and most preferably ≤0.05.

In another preferred embodiment, the low activity of the mitochondria permeability transition pore means that the ratio (A1/A0) of the activity level or expression level A1 of the mitochondria permeability transition pore in a cell (such as cancer cell) to the activity level or expression level A0 of the mitochondria permeability transition pore in a normal cell (the same type of cell) is ≤0.8, preferably ≤0.7, more preferably ≤0.6, more preferably ≤0.5, more preferably ≤0.4, more preferably ≤0.3, more preferably ≤0.2, more preferably ≤0.1, most preferably ≤0.05.

In another preferred embodiment, the cancer is not sensitive to conventional radiotherapy and chemotherapy.

In another preferred embodiment, the cancer is recurrent or metastatic cancer.

In another preferred embodiment, the cancer is cancer stem cell.

In another preferred embodiment, the cancer is a cancer with stem cell properties.

In another preferred embodiment, the cancer comprises but is not limited to cancer containing FGFR3-TACC3 fusion gene, cancer with high expression of BACH1 transcription factor, cancer with high expression of myc protein, cancer with kras mutation, cancer with glycolysis deficiency, cancer with TP53 gene mutation, cancer with BRAF gene mutation, cancer with CDKN2A gene mutation, cancer with PTEN gene mutation, cancer with CDKN2C gene mutation, cancer with CTNNB1 gene mutation, cancer with EGFR gene mutation, cancer with NRAS gene mutation, cancer with STK11 gene mutation, cancer with BARF gene mutation, cancer with SMAD4 gene mutation, cancer with MAP2K4 gene mutation, cancer with PBXW7 gene mutation, cancer with KDM6A gene mutation, cancer with BRCA2 gene mutation, cancer with BRCA1 gene mutation, cancer with RB1 gene mutation, cancer with CDH1 gene mutation, cancer with PIK3CA gene mutation, cancer with NPM1 gene mutation, cancer with DNMT3A R882C mutation, and combinations thereof.

Representatively, the brain cancer comprises but is not limited to glioma, medulloblastoma.

Preferably, the glioma comprises glioma with CDKN2A, PTEN and/or CDKN2C gene mutation.

Preferably, the glioma is glioblastoma.

Preferably, the brain cancer is brain glial cell carcinoma.

Preferably, the brain cancer is brain glioblastoma.

Preferably, the glioblastoma is glioblastoma multiforme.

Preferably, the brain cancer is medulloblastoma.

Preferably, the medulloblastoma is cerebellar medulloblastoma.

Representatively, the breast cancer comprises but is not limited to triple negative breast cancer, breast ductal adenocarcinoma, breast squamous cell carcinoma, metastatic breast cancer, and combinations thereof.

As used herein, the term “metastatic breast cancer” refers to breast cancer that originates from the breast and metastasizes to other organs in the body.

Preferably, the breast ductal adenocarcinoma is invasive breast ductal adenocarcinoma.

Preferably, the invasive breast ductal adenocarcinoma comprises invasive breast ducal adenocarcinoma with PTEN, RB1 and/or TP53 gene mutation.

Preferably, the breast squamous cell carcinoma is breast acantholysis squamous cell carcinoma.

Preferably, the breast acantholysis squamous cell carcinoma is breast TNM IIB stage 2 primary acantholysis squamous cell carcinoma.

Preferably, the breast acantholysis squamous cell carcinoma comprises breast acantholysis squamous cell carcinoma with CDKN2A STK11, KDM6A and/or TP53 gene mutation.

Preferably, the metastatic breast cancer comprises breast cancer with CDH1 and/or PIK3CA gene mutation.

Representatively, the liver cancer is anaplastic and lowly differentiated liver cancer.

Preferably, the liver cancer comprises liver cancer with CTNNB1 and/or NRAS gene mutation.

Preferably, the liver cancer is grade II-III/IV liver cancer.

Representatively, the melanoma is selected from the group consisting of multidrug-resistant melanoma, malignant melanoma, and combinations thereof.

Preferably, the malignant melanoma is malignant melanoma with inguinal lymph node metastasis.

Preferably, the malignant melanoma comprises malignant melanoma with BRAF, CDKN2A and/or STK11 gene mutation.

Preferably, the malignant melanoma is metastatic malignant melanoma, and combinations therefore.

Representatively, the leukemia comprises but is not limited to myeloid leukemia, T lymphocyte leukemia, and combinations thereof.

Preferably, the myeloid leukemia is acute myeloid leukemia (AML).

Preferably, the lymphocyte leukemia is acute lymphocyte leukemia.

Preferably, the acute myeloid leukemia is M4 grade acute myeloid leukemia.

Preferably, the acute myeloid leukemia is FAB M4 grade AML acute myeloid leukemia.

Preferably, the FAB M4 grade acute myeloid leukemia comprises FAB M4 grade acute myeloid leukemia with NPM1 and/or DNMT3A R882C gene mutation.

Representatively, the lung cancer comprises but is not limited to small cell lung cancer, non-small cell lung cancer, and combinations thereof.

Representatively, the brain cancer is brain glial cell carcinoma.

Representatively, the lymphoma comprises but is not limited to B cell lymphoma, mononuclear cell lymphoma, T cell lymphoma, and combinations thereof.

Preferably, the cell lymphoma is cutaneous T cell lymphoma.

Preferably, the lymphoma is Non-Hodgkin's lymphoma (NHL).

Preferably, the Non-Hodgkin's lymphoma (NHL) is cutaneous T cell lymphoma.

As used herein, the term “cutaneous T cell lymphoma (CTCL)” is a type of non-Hodgkin's lymphoma (NHL), caused by clonal proliferation of T lymphocytes originating from the skin and is composed of a group of diseases with different clinical manifestations, histological features, course and prognosis.

Representatively, the pancreatic cancer comprises but is not limited to pancreatic ductal adenocarcinoma, liver metastatic pancreatic cancer, and combinations thereof.

Preferably, the pancreatic ductal adenocarcinoma comprises pancreatic ductal adenocarcinoma with TP53 gene mutation.

Representatively, the kidney cancer comprises but is not limited to kidney rhabdoid carcinoma, kidney smooth muscle carcinoma, kidney cell adenocarcinoma, and combinations thereof.

Preferably, the kidney cell adenocarcinoma is metastatic kidney cell adenocarcinoma.

Preferably, the kidney cell adenocarcinoma is primary kidney cell adenocarcinoma.

Preferably, the intestinal cancer is colorectal adenocarcinoma.

Preferably, the colorectal adenocarcinoma comprises but is not limited to Dukes' type B colorectal adenocarcinoma, Dukes' type C, grade IV colorectal adenocarcinoma, and combinations thereof.

Preferably, the Dukes' type C, grade IV colorectal adenocarcinoma comprises Dukes' type C, grade IV colorectal adenocarcinoma with CTNNB1, EGFR and/or FBXW7 gene mutations.

Representatively, the colorectal cancer comprises but is not limited to colorectal adenocarcinoma.

In another preferred embodiment, the cancer is cancer with upregulation of the oxidative phosphorylation pathway of mitochondria and/or low activity of the mitochondria permeability transition pore in cancer cell.

Preferably, the upregulation of the oxidative phosphorylation pathway of mitochondria means that the ratio (E1/E0) of the level or expression E1 of the oxidative phosphorylation pathway of mitochondria in a cell (such as cancer cell) to the level or expression E0 of the oxidative phosphorylation pathway of mitochondria in a normal cell (the same type of cell) is ≥1.2, preferably ≥1.5, more preferably ≥2, more preferably ≥3, more preferably ≥5.

Preferably, the low activity of the mitochondria permeability transition pore means that the ratio (A1/A0) of the activity level or expression level A1 of the mitochondria permeability transition pore in a cell (such as cancer cell) to the activity level or expression level A0 of the mitochondria permeability transition pore in a normal cell (the same type of cell) is ≤0.8, preferably ≤0.7, more preferably ≤0.6, more preferably ≤0.5, more preferably ≤0.4, more preferably ≤0.3, more preferably ≤0.2, more preferably ≤0.1, most preferably ≤0.05.

Anticancer Drug

In the present invention, anticancer drug can be the compound of formula I, or an optical isomer thereof, or a racemate thereof, or a solvate thereof, or a pharmaceutically acceptable salt thereof, and/or other anticancer drugs, and combinations thereof.

In a preferred embodiment, the other anticancer drugs comprise but are not limited to immunotherapy drugs, chemotherapeutic drugs that block DNA synthesis, anticancer drugs that promote cell death, inhibitors targeting proteasome, and combinations thereof.

Representatively, the immunotherapy drugs comprise but are not limited to PD1/PDL1, CTLA-4, and combinations thereof.

Representatively, the chemotherapeutic drugs that block DNA synthesis comprise but are not limited to tegafur, fluorouracil, oxaliplatin, temozolomide, and combinations thereof.

Representatively, the anticancer drug that promotes cell death is Bcl-2 small molecule inhibitor (e.g., Venetoclax).

Representatively, the inhibitor targeting proteasome is Bortezomib.

Viral Infection

The compound of the present invention have significant therapeutic effect on viral infection and/or its related diseases.

In another preferred embodiment, the virus comprises but is not limited to influenza virus, parainfluenza virus, cytomegalovirus adenovirus, rhinovirus, coronavirus, coxsackie virus, eko virus, chickenpox, rubella, measles virus, respiratory syncytial virus.

In another preferred embodiment, the virus is coronavirus.

In another preferred embodiment, the coronavirus comprises but is not limited to α coronavirus, β coronavirus, and combinations thereof.

In another preferred embodiment, the coronavirus comprises but is not limited to HCoV-229E, HCoV-OC43, SARS-CoV, HCoV-NL63, HCoV-HKU1, MERS-CoV, 2019-nCov, and combinations thereof.

In another preferred embodiment, the coronavirus comprises but is not limited to 2019 novel Coronavirus (2019-nCov), SARS virus, MERS virus, and combinations thereof.

In another preferred embodiment, the coronavirus comprises but is not limited to HCoV-229E, HCoV-OC43, SARS-CoV, HCoV-NL63, HCoV-HKU1, MERS-CoV, 2019-nCov, and combinations thereof.

As used herein, the term “2019 novel Coronavirus (2019-nCoV)”, “novel coronavirus-infected pneumonia”, “novel coronavirus pneumonia” and “Novel Coronavirus Pneumonia (NCP)” are used interchangeably.

As used herein, the viral infection related disease is preferably pneumonia, pulmonary fibrosis.

As used herein, “pneumonia” is a common clinical pathological change in the lungs, and its symptoms comprises cough, shortness of breath, chest tightness, fatigue, fever, and dyspnea in severe cases. Increased exudation, interstitial thickening, lung density change, such as multiple small patch shadows, and multiple ground-glass shadow in both lungs can exist on the images, and lung consolidation can occur in severe cases. On the blood picture, the total number and classification of white blood cells can be changed. Biochemically, stress reaction can be occurred, such as changes in C-reactive protein/erythrocyte sedimentation rate/procalcitonin, liver enzymes, muscle enzymes, and myoglobin. The etiology is persistent, and pneumonia can be transformed to pulmonary fibrosis.

As used herein, “pulmonary fibrosis” is a disease characterized by diffuse pneumonia and alveolar structural disorder that ultimately lead to pulmonary interstitial fibrosis, and a severe pathological feature shared by a class of clinically known interstitial lung diseases. Interstitial lung disease can comprise seven major categories as follows: primary lung disease, accompanying systemic rheumatic disease, drug or radiation treatment related disease, accompanying environmental or occupational disease, accompanying pulmonary vasculogenesis disease alveolax stasis disease and hereditary disease. Pulmonary fibrosis can be divided into two categories as follows: idiopathic and secondary based on the causes its common feature is that the normal alveolar structure is first damaged by inflammation caused by various reasons, ie, alveolitis is caused, then collagen scar tissue is accumulated to repair the injury, that is, fibrosis occurs and the lung tissue gradually loses its normal respiratory function, resulting in dyspnea, hypoxia and other symptoms, finally leading to respiratory failure. The incidence rate of pulmonary fibrosis, especially idiopathic pulmonary fibrosis, caused by various causes has been increasing in recent years.

Mitochondria Permeability Transition Pore Inhibitor and the Use Thereof

In the present invention, the mitochondria permeability transition pore inhibitor comprises but is not limited to Cyclosporin A, CyP-D protein inhibitor, peroxide scavenger, and combinations thereof.

Representatively, the CyP-D protein inhibitor is SfA, BKA and ADP (small molecule that regulates the activity of ANT protein).

Representatively, the peroxide scavenger comprises but is not limited to Propofol, pyruvate, MCI-186, and combinations thereof.

The present invention further provides a use of the mitochondria permeability transition pore inhibitor for enhancing the anticancer effect of anticancer drugs.

A Use of the Compound of the Present Invention

The present invention provides the compound of formula I, or an optical isomer thereof, or a racemate thereof, or a solvate thereof, or a pharmaceutically acceptable salt thereof for one or more uses comprising but not limited to the following group: (a) inhibiting the oxidative phosphorylation pathway of mitochondria; (b) preventing and/or treating diseases associated with oxidative phosphorylation pathway of mitochondria; and (c) preventing and/or treating cancers.

In a preferred embodiment, the disease associated with oxidative phosphorylation pathway of mitochondria is cancer.

The present invention further provides a method for non-therapeutically and non-diagnostically inhibiting oxidative phosphorylation pathway of mitochondria in vitro, which comprises contacting the oxidative phosphorylation pathway of mitochondria or cell expressing the oxidative phosphorylation pathway of mitochondria with the compound of formula I, or an optical isomer thereof, or a racemate thereof, or a solvate thereof, or a pharmaceutically acceptable salt thereof of the present invention, thereby inhibiting oxidative phosphorylation pathway of mitochondria.

The present invention further provides a method for non-therapeutically and non-diagnostically inhibiting cancer cell in vitro, which comprises contacting the cancer cell with the compound of formula I, or an optical isomer thereof, or a racemate thereof, or a solvate thereof, or a pharmaceutically acceptable salt thereof of the present invention, thereby inhibiting cancer cell.

In the present invention, the method for non-therapeutically and non-diagnostically inhibiting oxidative phosphorylation pathway of mitochondria and inhibiting cancer cell in vitro can be used for drug screening, quality control and other purposes. For example, by contacting the oxidative phosphorylation pathway of mitochondria or the cell expressing the oxidative phosphorylation pathway of mitochondria, or cancer cell with the compound of formula I, or an optical isomer thereof, or a racemate thereof, or a solvate thereof, or a pharmaceutically acceptable salt thereof of the present invention, the drug that can inhibit the oxidative phosphorylation pathway of mitochondria, the cell expressing the oxidative phosphorylation pathway of mitochondria or the cancer cell can be used as drug candidate, and animal experiments and clinical trials are further performed to investigate the therapeutic effect of drug candidate on the oxidative phosphorylation pathway of mitochondria, cell expressing the oxidative phosphorylation pathway of mitochondria, or cancer cell.

The present invention further provides a method for (a) inhibiting the oxidative phosphorylation pathway of mitochondria; (b) preventing and/or treating diseases associated with oxidative phosphorylation pathway of mitochondria; and/or (c) preventing and/or treating cancers, which comprises administering the compound of formula I, or an optical isomer thereof, or a racemate thereof, or a solvate thereof, or a pharmaceutically acceptable salt thereof, or the composition according to the present invention to a subject in need, thereby (a) inhibiting the oxidative phosphorylation pathway of mitochondria; (b) preventing and/or treating diseases associated with oxidative phosphorylation pathway mitochondria; and/or (c) preventing and/or treating cancers.

In another preferred embodiment, the subject is human and non-human mammals (rodent, rabbit, monkey, livestock, dog, cat, and the like).

Pharmaceutical Composition or Preparation, Active Ingredient Combination, Medical Kit and Administration Method

The invention provides a composition or preparation, an active ingredient combination and a medical kit, the composition or preparation, active ingredient combination and medical kit can be used for (a) inhibiting the oxidative phosphorylation pathway of mitochondria; (b) preventing and/or treating diseases associated with oxidative phosphorylation pathway of mitochondria; and/or (c) preventing and/or treating cancers.

Preferably, the composition of the present invention is pharmaceutical composition. The compositions of the present invention can comprise a pharmaceutically acceptable carrier.

The term “pharmaceutically acceptable carrier” refers to one or more compatible solid, semi-solid, liquid or gel fillers, which are suitable for use in humans or animals and must have sufficient purity and sufficiently low toxicity. The “compatible” means each ingredient of the pharmaceutical composition and active ingredient of the drug can be blended with each other without significantly reducing the efficacy.

It should be understood that the pharmaceutically acceptable carrier is not particularly limited in the present invention, the carrier can be selected from materials commonly used in the art, or can be obtained by a conventional method, or is commercially available. Some examples of pharmaceutically acceptable carriers are cellulose and its derivatives (such as methylcellulose, ethylcellulose, hydroxypropylmethylcellulose, sodium carboxymethylcellulose, etc.), gelatin, talc, solid lubricants (such as stearic acid, magnesium stearate), calcium sulfate, plant oil (such as soybean oil, sesame oil, peanut oil, olive oil, etc.), polyols (such as propylene glycol, glycerin, mannitol, sorbitol, etc.), emulsifier (such as Tween), wetting agent (such as sodium lauryl sulfate), buffer agent, chelating agent, thickener, pH regulator, transdermal enhancer, colorant, flavoring agent, stabilizer, antioxidant, preservative, bacteriostatic agent, pyrogen-free water, etc.

In a preferred embodiment, the composition or preparation, medical kit further comprises other anticancer drugs.

In vitro studies and in vivo administration (such as intratumoral administration), mitochondria permeability transition pore inhibitor can enhance the therapeutic effect of antitumor drugs by reducing mPTP activity and up-regulating oxidative phosphorylation pathway of mitochondria in tumor cells. Therefore, anti-tumor drug and mPTP inhibitor can play a synergistic anti-tumor effect.

The present invention provides an active ingredient combination, composition and medical kit comprising antitumor drug and mPTP inhibitor for synergistic antitumor effect.

The invention provides an active ingredient combination, the active ingredient combination comprise:

(1) a first active ingredient, the first active ingredient is anticancer drug; and

(2) a second active ingredient, the second active ingredient is mitochondria permeability transition pore inhibitor.

In another preferred embodiment, at least one active ingredient is independent in the active ingredient combination.

In another preferred embodiment, the first active ingredient and the second active ingredient are independent of each other in the active ingredient combination.

The invention provides a composition comprising:

(1) a first active ingredient, the first active ingredient is anticancer drug; and

(2) a second active ingredient the second active ingredient is mitochondria permeability transition pore inhibitor.

In another preferred embodiment, the composition is a pharmaceutical composition.

In another preferred embodiment, the pharmaceutical composition further comprises a pharmaceutically acceptable carrier.

In another preferred embodiment, the content of the first active ingredient is 0.01-99.99 wt %, preferably 0.1-99.9 wt %, more preferably 1-99 wt %, more preferably 10-99 wt %, most preferably 20-99 wt %, based on the total weight of the active ingredient of the composition.

In another preferred embodiment, the content of the second active ingredient is 0.01-99.99 wt %, preferably 0.1-99.9 wt %, more preferably 1-99 wt %, more preferably 10-99 wt %, and most preferably 20-99 wt %, based on the total weight of the active ingredients of the composition.

The invention provides a medical kit, the medical kit comprises:

(A) a first preparation comprising a first active ingredient, the first active ingredient is anticancer drug; and

(B) a second preparation comprising a second active ingredient, the second active ingredient is mitochondria permeability transition pore inhibitor.

In another preferred embodiment, the medical kit further comprises user's manual.

In another preferred embodiment, the first preparation and the second preparation are independent of each other.

In another preferred embodiment, the first preparation and the second preparation are combined preparation.

In another preferred embodiment, the user's manual records that the first preparation and the second preparation are used in combination to enhance the anti-tumor activity of the anticancer drug.

In another preferred embodiment, the method for use in combination is to administer the second preparation comprising the mitochondria permeability transition pore inhibitor first, and then administer the anticancer drug.

Preferably, the molar ratio of the first active ingredient to the second active ingredient is 0.01-600:1, preferably 0.05-500:1, more preferably 0.1-400:1, more preferably 0.2-200:1, more preferably 0.5-100:1, more preferably 0.5-80:1, most preferably 1-50:1 in the active combination, composition and/or medical kit of the present invention.

In the present invention, the dosage form of composition or preparation comprises but is not limited to oral preparation, injection preparation and external preparation.

Representatively, the dosage form of the composition or preparation comprises but is not limited to tablet, injection, infusion, ointment, gel solution, microsphere, and film.

Typically, the injection is intratumoral injection.

The pharmaceutical preparation should be matched with the mode of administration. Preferred mode of administration is oral administration, injection administration ((e.g. intratumoral injection), and when used, a therapeutically effective amount of the drug is administered to a subject in need (e.g. human or non-human mammal). As used herein, the term “therapeutically effective amount” refers to an amount that has a function or activity in human and/or animal and is acceptable to human and/or animal. It should be understand in the art that the “therapeutically effective amount” can vary depending on the form of the pharmaceutical composition, the route of administration, the excipients of the drug, the severity of the disease, and the combination with other drugs, etc.

In a mode of administration, the safe and effective daily dose of the first active ingredient is usually at least about 0.1 mg, and does not exceed about 2500 mg in most case. Preferably, the dose is 1 mg to 500 mg. The safe and effective amount of the second active ingredient is usually at least about 0.01 mg, and does not exceed about 2500 mg in most case. Preferably, the dose range is 0.1 mg to 2500 mg. Of course, the specific dose should also take into account the route of administration, the patients health and other factors, which are within the skill range of skilled doctors.

The Main Advantages of the Present Invention Comprise:

The present invention firstly finds that the compound of formula I can efficiently and safely inhibit the oxidative phosphorylation pathway of mitochondria, and prevent and/or treat diseases (such as cancer) associated with oxidative phosphorylation pathway of mitochondria, in particular, the compound of formula I has significant inhibitory effect on tumor cells with the upregulation of oxidative phosphorylation pathway of mitochondria or low activity of mPTP.

The present invention will be further illustrated below with reference to the specific examples. It should be understood that these examples are only to illustrate the invention but are not to limit the scope of the invention. The experimental methods with no specific conditions described in the following examples are generally performed under the conventional conditions, or according to the manufacturer's instructions. Unless indicated otherwise, parts and percentage are calculated by weight.

### EXAMPLE

The structure of the Compound SJ2858 (baicalein) and Compound SJ2775 (baicalin) were shown below:

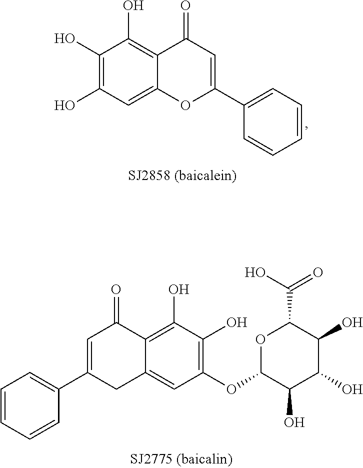 [SMILES: O=c1cc(-c2ccccc2)oc2cc(O)c(O)c(O)c12 ; O=C1C=C(c2ccccc2)Cc2cc(O[C@@H]3O[C@H](C(=O)O)[C@@H](O)[C@H](O)[C@H]3O)c(O)c(O)c21]

Daoy cell is human medulloblastoma cell, NCI-H82 cell is small cell lung cancer cell.

### Example 1

Investigating the Activity of Mitochondria Permeability Transition Pore in Daoy Cell and NCI-H82 Cell

1.1 Experimental Background:

The mitochondria permeability transition pore (mPTP) was a non-specific channel on the inner membrane of mitochondria, which could allow small molecules with molecular less than 1.5 KD to pass freely, and its activity was affected by peroxides (such as H₂O₂), pH and calcium ions in mitochondria. Some cells had active mitochondria permeability transition pore, while some cells had inactive mitochondria permeability transition pore. For cells with active mitochondria permeability transition pore, adding peroxide (such as H₂O₂) could increase the activity of mitochondria permeability transition pore, resulting in a decrease in mitochondria membrane potential. For cells with inactive mitochondria permeability transition pole, adding peroxide (such as H₂O₂) had no significant effect on the activity of the mitochondria permeability transition pore, and the mitochondria membrane potential had no significant change. Based on this principle, whether the mPTP was active or inactive in specific cell could be determined by measuring the change of potential difference of the mitochondria membrane under peroxide stimulation.

1.2 Experimental Method and Result

Daoy cell and NCI-H82 cell were cultured in DMEM containing 10% FBS (plus p/s), and then 1.5 μM Cyclosporin A (C_{S}A, C_{S}A could effectively inhibit the activity of mPTP (mitochondria permeability transition pore)) was added to the medium, and the cell cultured in DMEM without the addition of Cyclosporin A (C_{S}A) were used as blank control. The mitochondria membrane potential difference was detected by Tetramethylrhodamine (TMRM), the high fluorescence intensity of TMRM represented the high membrane potential difference.

TABLE 1
Relative TMRM signal intensity (%) in different groups

| Cell | Daoy cell | | | | NCI-H82 cell | | | |
|---|---|---|---|---|---|---|---|---|
| CsA | − | − | + | + | − | − | + | + |
| H₂O₂ | − | + | − | + | − | + | − | + |
| mean value | 100 | 82 | 125 | 121 | 118 | 119 | 120 | 118 |
| Standard | 7 | 11 | 14 | 11 | 5 | 2 | 6 | 8 |
| deviation | | | | | | | | |
| Repeat times | 3 | 3 | 3 | 3 | 3 | 3 | 3 | 3 |
| P value | | <0.05 | <0.01 | | | | <0.05 | <0.05 |

Remarks: “+” represented existence, “−” represented none.

It could be seen from Table 1 that the membrane potential of Daoy cell was down-regulated under the action of H₂O₂, and the down-regulation could be reversed by C_{S}A, a mitochondria permeability transition pore inhibitor, indicating that the mitochondria permeability transition pore was active in Daoy cell. The membrane potential of NCI-H82 cell was not affected by H₂O₂ and C_{S}A, indicating that mPTP (mitochondria permeability transition pore) was inactive in NCI-H82 cell. Therefore, Table 1 shown that the mitochondria permeability transition pore was active in Daoy cell, while the mitochondria permeability transition pore was inactive in NCI-H82 cell.

### Example 2

Investigating the Inhibitory Effect of Compound SJ2858 and Compound SJ2775 on Oxidative Phosphorylation Pathway of Mitochondria

2.1 Experimental Background

The oxygen required by cell was mainly consumed in the mitochondria respiratory chain, so the measurement of cellular oxygen consumption (OCR, oxygen consumption rate) could directly reflect the activity of the oxidative phosphorylation pathway of mitochondria. In this experiment, the XFe metabolic analysis system was used to measure the oxygen consumption of NCI-H82 cell in the presence or absence of Compound SJ2858 (baicalein) and Compound SJ2775 (baicalin).

2.2 Experimental Method and Result

Rotenone (abbreviated as RO, 0.5 μM) is a known inhibitor of mitochondria oxidative phosphorylation pathway complex I, Antimycin A (abbreviated as AA, 0.5 μM) is a known inhibitor of mitochondria oxidative phosphorylation pathway complex III. Compound SJ2858, 73.2 μM; Compound SJ2775, 118.2 μM.

Specific Steps:

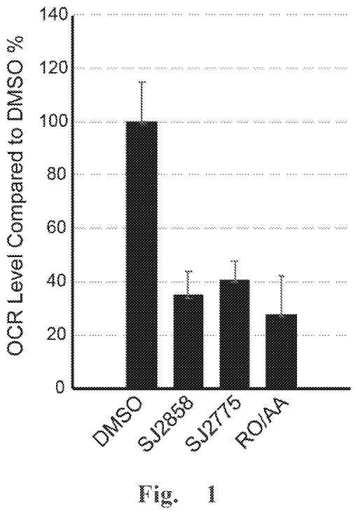

NCI-H82 cells were cultured in DMEM medium containing 10% FBS (plus p/s), and the detection of the inhibition of cellular oxygen consumption was completed within half an hour after the addition of complex inhibitors (RO and AA, RO/AA), Compound SJ2858 and Compound SJ2775, respectively, and the addition of dimethyl sulfoxide (DMSO) was used as the blank control group. The experimental results were shown in FIG. 1.

The FIG. 1 showed that the Compound SJ2858 and Compound SJ2775 could significantly inhibit the oxygen consumption of NCI-H82 cell, and had similar inhibitory effect to the positive control RO/AA, indicating that Compound SJ2858 and Compound SJ2775 could significantly inhibit the activity of the oxidative phosphorylation pathway of mitochondria.

### Example 3

The Inhibitory Activity of Compound SJ2858 and Compound SJ2775 on the Growth of Cancer Cell were Regulated by mPTP

3.1 Experimental Background

Cell viability was detected by using the Promega CellTiter-Glo kit, the kit reflected cell viability by directly measuring intracellular ATP content. In this experiment, Daoy cell with active mitochondria permeability transition pore and NCI-H82 cell with inactive mitochondria permeability transition pore were used to detect the IC50 value of cell viability inhibition of Compound SJ2858 and Compound SJ2775 on Daoy cell and Daoy cell+mPTP inhibitor (Cyclosporin A, CSA, 1.5 μM, Sigma) and NCI-H82.

3.2 Experimental Method and Result

Daoy cell and NCI-H82 cell were cultured in DMEM containing 10% FBS (plus p/s). The 50% inhibitory dose IC₅₀ of Compound SJ2858 and Compound SJ2775 was measured in two cells (Daoy cell and NCI-H82 cell) under three conditions (Daoy cell without CSA, Daoy cell with CSA and NCI-H82 cell without CSA). The experimental results are shown in Table 2 and table 3:

TABLE 2
the inhibitory effect of Compound SJ2S58 on Daoy cell without
CSA, Daoy cell with CSA, and NCI-H82 cell without CSA

| Treatment | Daoy cell | Daoy cell | NCI-H82 cell |
|---|---|---|---|
| group | without CSA | with CSA | without CSA |
| IC50 | 35.2 μM | 13.2 μM | 15.6 μM |

TABLE 3
the inhibitory effect of Compound SJ2775 on Daoy cell without
CSA, Daoy cell with CSA, and NCI-H82 cell without CSA

| Treatment | Daoy cell | Daoy cell | NCI-H82 cell |
|---|---|---|---|
| group | without CSA | with CSA | without CSA |
| IC50 | >100 μM | 45.4 μM | 38.6 μM |

The Table 2 showed that the Compound SJ8858 had significant inhibitory effect on mPTP-inactive cell (NCI-H82, and Daoy+mPTP inhibitor CSA), the 50% inhibitory dose IC50 was 15.6 μM and 13.2 μM, respectively, while the inhibitory effect of the Compound SJ2858 on the Daoy cell with active mPTP was relatively low, the 50% inhibitory dose IC50 was 35.2 μM.

The Table 3 showed that the Compound SJ2775 had significant inhibitory effect on mPTP inactive cell (NCI-H82, and Daoy+mPTP inhibitor CSA), the 50% inhibitory dose IC50 was 38.6 μM and 45.4 μM, respectively, while the inhibitory effect of the Compound SJ2775 on the Daoy cell with active mPTP was relatively low, the 50% inhibitory dose IC50 was >100 μM.

It could be seen from the results of example 3 that the Compound SJ2858 and Compound SJ2775 had inhibitory effect on Daoy cell and NCI-H82 cell, especially on NCI-H82 cell with inactive mPTP. In addition, when the mPTP activity of Daoy cell was decreased, the inhibitory effect of Compound SJ2858 and Compound SJ2775 on the cell could be significantly increased, indicating that decreasing the mPTP activity of cell could significantly enhance the antitumor activity of Compound SJ2858 and Compound SJ2775, the Compound SJ2858 and Compound SJ2775 had more significant inhibitory effect on mPTP-inactive cell.

### Example 4

Investigating the Inhibitory Effect of Compound SJ2858 on Various Tumor Cell Lines

Cell viability was measured by using the Promega CellTiter-Glo kit, the kit reflected cell viability by directly measuring intracellular ATP content. Different cancer cells were cultured in culture medium, and the 50% inhibitory concentration IC50 of the compound SJ2858 was measured by Promega CellTiter-Glo kit. The name source and culture condition of each tumor cell line were as follows:

Cell line Gp2D (ECACC, No. 95090714) was cultured in DMEM medium containing 10% fetal bovine serum (+P/S);

Cell line U-937 (ATCC, No. CRL-1593.2) was cultured in RPMI1640 medium containing 10% fetal bovine serum (+P/S);

Cell line A-375 (ATCC, No. CRL-1619) was cultured in DMEM medium containing 10% fetal bovine serum (+P/S);

Cell line SNU-398 (ATCC, No. CRL-2233) was cultured in RPMI1640 medium (+P/S); containing 10% fetal bovine serum (+P/S);

Cell line NCI-H1048 (ATCC, No. CRL-5853) was cultured in HITES medium containing 5% fetal bovine serum (+P/S);

Cell line HCC15 (KCLB, No. 70015) was cultured in RPMI1640 medium containing 10% fetal bovine serum (+P/S);

Cell line ATN-1 (RIKEN, No. RBRC-RCB1440) was cultured in RPMI1640 medium containing 10% fetal bovine serum and 0.1 mM NEAA (+P/S);

Cell line Jurkat, Clone E6-1 (ATCC, No. TIB-152) was cultured in RPMI1640 medium containing 10% fetal bovine serum (+P/S);

Cell line MIA PaCa-2 (ATCC, No. CRL-1420) was cultured in DMEM medium containing 10% fetal bovine serum (+P/S);

Cell line D283 Med (ATCC, No. HTB-185) was cultured in EMEM medium containing 10% fetal bovine serum (+P/S);

Cell line BT-549 (ATCC, No. HTB-122) was cultured in RPMI1640 medium containing 10% fetal bovine serum and 0.023 IU/ml human insulin (+P/S);

Cell line 22RV1 (ATCC, No. CRL-2505) was cultured in RPMI1640 medium containing 10% fetal bovine serum (+P/S);

Cell line H9 (ATCC, No. HTB-176) was cultured in RPMI1640 medium containing 10% fetal bovine serum (+P/S);

Cell line G-401 (ATCC, No. CRL-1441) was cultured in McCoy's 5a medium containing 10% fetal bovine serum (+P/S);

Cell line HCC1806 (ATCC, No. CRL-2335) was cultured in RPMI1640 medium containing 10% fetal bovine serum (+P/S);

Cell line OCI-LY-19 (DSMZ, code ACC-528) was cultured in 80-90% alpha-MEM medium containing 10-20% h.i. FBS (+P/S);

Cell line MDA-MB-453 (ATCC, No. HTB-131) was cultured in Leibovitz's L-15 medium containing 10% fetal bovine serum (+P/S);

Cell line SU-DHL-2 (ATCC, No. CRL-2956) was cultured in RPMI1640 medium containing 10% fetal bovine serum (+P/S);

Cell line G-402 (ATCC, No. CRL-1440) was cultured in McCoy's 5a medium containing 10% fetal bovine serum (+P/S);

Cell line CCRF-CEM (ATCC, No. CCL-119) was cultured in RPMI1640 medium containing 10% fetal bovine serum (+P/S);

Cell line HH (ATCC, No. CRL-2105) was cultured in RPMI1640 medium containing 10% fetal bovine serum (+P/S);

Cell line OCI-AML-3 (DSMZ, No. ACC-582) was cultured in RPMI1640 medium containing 20% fetal bovine serum (+P/S);

Cell line OCI-AML-4 (DSMZ, No. ACC-729) was cultured in alpha-MEM medium containing 20% fetal bovine serum and 20% volume fraction of 5637 cell line adjusted medium (+P/S);

Cell line OCI-AML-5 (DSMZ, No. ACC-247) was cultured in alpha-MEM medium containing 20% fetal bovine serum and 20% volume fraction of 5637 cell line adjusted medium (+P/S);

Cell line GAK (JCRB, No. JCRB0180) was cultured in Ham's F12 medium containing 20% fetal bovine serum (+P/S);

Cell line CHL-1 (ATCC, No. CRL-9446) was cultured in DMEM medium containing 10% fetal bovine serum (+P/S);

Cell line NCI-H1155 (ATCC, No. CRL-5818) was cultured in serum-free ACL-4 medium (+P/S);

Cell line LS 180 (ATCC, No. CL-187) was cultured in EMEM medium containing 10% fetal bovine serum (+P/S);

Cell line GB-1 (JCRB, No. IFO50489) was cultured in DMEM medium containing 10% fetal bovine serum (+P/S);

Cell line 786-O (ATCC, No. CRL-1932) was cultured in RPMI1640 medium containing 10% fetal bovine serum (+P/S);

Cell line SF126 (JCRB, No. IFO50286) was cultured in EMEM medium containing 10% fetal bovine serum (+P/S);

Cell line ACHN (ATCC, No. CRL-1611) was cultured in EMEM medium containing 10% fetal bovine serum (+P/S);

Cell line COLO 320HSR (ATCC, No. CCL-220.1) was cultured in RPMI1640 medium containing 10% fetal bovine serum (+P/S);

Cell line U-87MG (ATCC, No. HTB-14) was cultured in EMEM medium containing 10% fetal bovine serum (+P/S);

Cell line WSU-DLCL2 (DSMZ, No. ACC-575) was cultured in RPMI1640 medium containing 10% fetal bovine serum (+P/S);

Cell line SNU-449 (ATCC, No. CRL-2234) was cultured in RPMI1640 medium containing 10% fetal bovine serum (+P/S);

Cell line C3A (ATCC, No. CRL-10741) was cultured in EMEM medium containing 10% fetal bovine serum (+P/S);

Cell line NCI-H1793 (ATCC, No. CRL-5896) was cultured in HITES medium containing 5% fetal bovine serum (+P/S);

Cell line DU 145 (ATCC, No. HTB-81) was cultured in EMEM medium containing 10% fetal bovine serum (+P/S);

Cell line G-361 (ATCC, No. CRL-1424) was cultured in McCoy's 5a medium containing 10% fetal bovine serum (+P/S).

The inhibitory effect of the compound SJ2858 on various tumor cells was shown in Table 4.

TABLE 4
the inhibitory effect of the Compound
SJ2858 on various tumor cells

| | Cell line | Cancer | IC50(μM) |
|---|---|---|---|
| 1 | Gp2D | intestinal cancer | 14.9 |
| 2 | U-937 | lymphoma | 39.2 |
| 3 | A-375 | melanoma | 10.6 |
| 4 | SNU-398 | liver cancer | 13.0 |
| 5 | NCI-H1048 | lung cancer | 15.0 |
| 6 | HCC15 | lung cancer | 18.9 |
| 7 | ATN-1 | leukemia | 25.4 |
| 8 | Jurkat, Clone E6-1 | leukemia | 14.2 |
| 9 | MIA PaCa-2 | pancreatic cancer | 19.7 |
| 10 | D283 Med | brain cancer | 12.9 |
| 11 | BT-549 | breast cancer | 18.1 |
| 12 | 22RV1 | prostate cancer | 25.9 |
| 13 | H9 | lymphoma | 19.0 |
| 14 | G-401 | kidney cancer | 12.3 |
| 15 | HCC1806 | breast cancer | 25.7 |
| 16 | OCI-LY-19 | lymphoma | 52.3 |
| 17 | MDA-MB-453 | breast cancer | 75.3 |
| 18 | SU-DHL-2 | lymphoma | 6.2 |
| 19 | G-402 | kidney cancer | 4.2 |
| 20 | CCRF-CEM | leukemia | 14.7 |
| 21 | HH | lymphoma | 34.0 |
| 22 | OCI-AML-3 | leukemia | 62.3 |
| 23 | OCI-AML-4 | leukemia | 24.6 |
| 24 | OCI-AML-5 | leukemia | 45.1 |
| 25 | GAK | melanoma | 23.1 |
| 26 | CHL-1 | melanoma | 9.5 |
| 27 | NCI-H1155 | lung cancer | 19.6 |
| 28 | LS 180 | intestinal cancer | 17.4 |
| 29 | GB-1 | brain cancer | 12.4 |
| 30 | 786-O | kidney cancer | 10.6 |
| 31 | SF126 | brain cancer | 14.0 |
| 32 | ACHN | kidney cancer | 17.7 |
| 33 | COLO 320HSR | intestinal cancer | 17.8 |
| 34 | U-87 MG | brain cancer | 19.0 |
| 35 | WSU-DLCL2 | lymphoma | 21.3 |
| 36 | SNU-449 | liver cancer | 26.9 |
| 37 | C3A | liver cancer | 27.0 |
| 38 | NCI-H 1793 | lung cancer | 35.2 |
| 39 | DU 145 | prostate cancer | 37.8 |
| 40 | G-361 | melanoma | 33.6 |

Remarks: A-375 was malignant melanoma with BRAF, CDKN2A gene mutation, SNU-398 was anaplastic and lowly differentiated liver cancer, NCI-H1048 was small cell lung cancer, HCC15 was non-small cell lung cancer, U-937 was monocyte lymphoma, ATN-1 was T cell leukemia, Jurkat, Clone E6-1 was acute T cell lymphocyte leukemia, MIA PaCa-2 was pancreatic cancer with TP53 gene mutation, D283 Med was cerebellar medulloblastoma, BT-549 was invasive breast ductal adenocarcinoma with PTEN, RB1, TP53 gene mutations, H9 was cutaneous T cell lymphoma, G-401 was kidney rhabdoid carcinoma, HCC1806 was breast TNM stage IIB grade 2 primary acantholysis squamous cell carcinoma with CDKN2A, STK11, KDM6A, TP53 gene mutations, OCI-LY-19 was B cell lymphoma, MDA-MB-453 was metastatic breast cancer with CDH, PIK3CA gene mutation, SU-DHL-2 was B cell lymphoma, G-402 was kidney smooth muscle carcinoma, CCRF-CEM was acute T lymphocyte leukemia, HH was cutaneous T cell lymphoma, OCI-AML-3 was FAB M4 grade AML acute myeloid leukemia with NPM1 and DNMT3A R882C gene mutation), OCI-AML-4 was M4 grade AML acute myeloid leukemia, OCI-AML -5 was M4 grade AML acute myeloid leukemia, GAK was malignant melanoma with inguinal lymph node metastasis, CHL-1 was malignant melanoma, NCI-H1155 was non-small cell lung cancer, LS 180 was Dukes' type B colorectal adenocarcinoma, 786-O was primary kidney cell adenocarcinoma, ACHN was metastatic kidney cell adenocarcinoma, COLO 320HSR was colorectal adenocarcinoma, GB-1 was brain glioblastoma, SF126 was glioblastoma multiforme, U-87 MG was malignant glioma with CDKN2A, PTEN and CDKN2C gene mutations, WSU-DLCL2 was B cell lymphoma, SNU-449 was grade II-III/IV hepatocellular carcinoma, C3A was hepatocellular carcinoma with CTNNB1 and NRAS gene mutation, NCI-H1793 was non-small cell lung cancer, G-361 was malignant melanoma with BRAF, CDKN2A, STK11 gene mutation.

The Table 4 showed that the Compound SJ2858 had excellent inhibitory effect on various tumor cells.

All documents mentioned in the present invention are incorporated herein by reference, as if each document is individually cited for reference. It should be understood that those skilled in the art will be able to make various changes or modifications to the present invention after reading the teachings of the present invention, which also fall within the scope of the claims appended hereto.

1-15. (canceled)

16. A method for (a) inhibiting the oxidative phosphorylation pathway of mitochondria: (b) preventing and/or treating diseases associated with oxidative phosphorylation pathway of mitochondria; and/or (c) preventing and/or treating cancers, which comprises administering a compound of formula I, or an optical isomer thereof, or a racemate thereof, or a solvate thereof, or a pharmaceutically acceptable salt thereof to a subject in need; 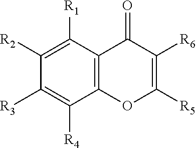
wherein,
R₁, R₂, R₃, R₄, R₅ and R₆ are each independently hydrogen, halogen, —CN, hydroxyl sulfhydryl, nitro, amino, —COOH, —CHO, substituted or unsubstituted C1-C12 alkyl, substituted or unsubstituted C3-C16 cycloalkyl, substituted or unsubstituted C1-C12 alkoxyl, substituted or unsubstituted C1-C12 alkylthio, substituted or unsubstituted 3-16 membered heterocycloalkyl, substituted or unsubstituted C6-C16 aryl, substituted or unsubstituted 3-16 membered heteroaryl, substituted or unsubstituted glycosyl-O—, substituted or unsubstituted glycosyl-S—, substituted or unsubstituted uronic acid group-O—, or substituted or unsubstituted uronic acid group-S—;
each “substituted” means that one or more (preferably 1, 2, 3, or 4) hydrogen atoms on the group are substituted by a substituent selected from the group consisting of C1-C8 alkyl, C3-C8 cycloalkyl, C1-C8 haloalkyl, C3-C8 halocycloalkyl, halogen, nitro, —CN, hydroxyl, sulfhydryl, amino, C1-C4 carboxyl, C2-C4 ester group, C2-C4 amido, C1-C8 alkoxyl, C1-C8 alkylthio, C1-C8 haloalkoxyl, C1-C8 haloalkylthio, C6-C12 aryl, 5-10 membered heteroaryl, 5-10 membered heterocycloalkyl;
the heterocyclic ring of the heterocycloalkyl and heteroaryl each independently contains 1-4 (preferably 1, 2, 3 or 4) heteroatoms selected from the group consisting of N, O and S.

17. The method of claim 16, wherein R₁ and R₂ are each independently hydrogen, hydroxyl or sulfhydryl;
R₃ is hydroxyl, sulfhydryl, substituted or unsubstituted C1-C4 alkoxyl, substituted or unsubstituted C1-C4 alkylthio, glucosyl-O—, glucuronic acid group-O—, galacturonic acid group-O—, mannuronic acid group-O—, iduronic acid group-O— or guluronic acid group-O—;
R₄ is hydrogen, methoxyl, or methylthio;
R₅ is substituted or unsubstituted phenyl, and the “substituted” means that one or more (preferably 1, 2, 3, or 4) hydrogen atoms on the phenyl are substituted by a substituent selected from the group consisting of hydroxyl, sulfhydryl; and/or
R₆ is hydrogen, hydroxyl or sulfhydryl.

18. The method of claim 16, wherein the compound of formula I is selected from the following group:

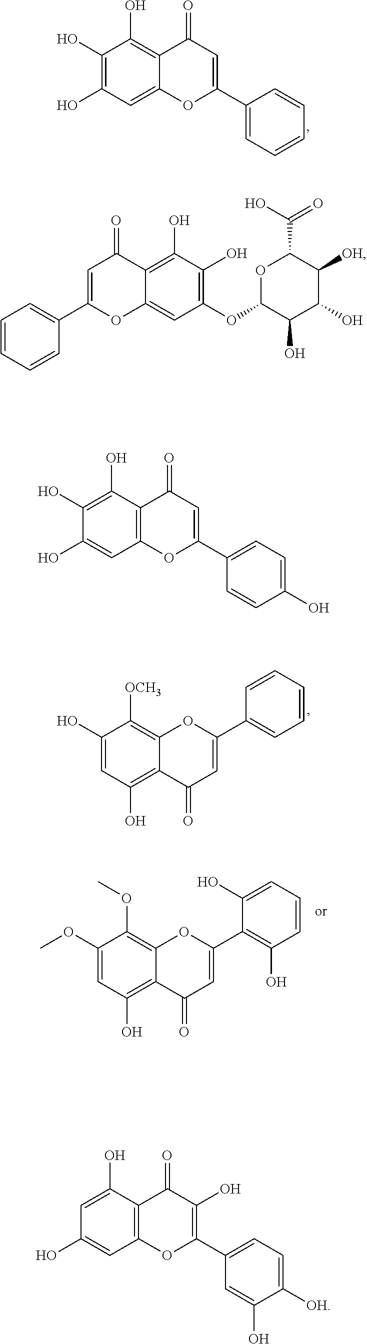 [SMILES: O=c1cc(-c2ccccc2)oc2cc(O)c(O)c(O)c12 ; O=C(O)[C@H]1O[C@@H](Oc2cc3oc(-c4ccccc4)cc(=O)c3c(O)c2O)[C@H](O)[C@@H](O)[C@@H]1O ; O=c1cc(-c2ccc(O)cc2)oc2cc(O)c(O)c(O)c12 ; COc1c(O)cc(O)c2c(=O)cc(-c3ccccc3)oc12 ; COc1cc(O)c2c(=O)cc(-c3c(O)cccc3O)oc2c1OC ; O=c1c(O)c(-c2ccc(O)c(O)c2)oc2cc(O)cc(O)c12]

19. The method of claim 16, wherein the diseases associated with oxidative phosphorylation pathway of mitochondria are selected from the group consisting of cancers, immune related diseases, neurodegenerative diseases, viral infection and/or its related diseases, and combinations thereof.

20. The method of claim 16, wherein the cancer is selected from the group consisting of lung cancer, pancreatic cancer, breast cancer, lymphoma, prostate cancer, brain cancer, leukemia, liver cancer, melanoma, intestinal cancer, kidney cancer, and combinations thereof.

21. The method of claim 20, wherein the brain cancer is selected from the group consisting of glioma, medulloblastoma;
the breast cancer is selected from the group consisting of triple negative breast cancer, breast ductal adenocarcinoma, breast squamous cell carcinoma, metastatic breast cancer, and combinations thereof;
the liver cancer is anaplastic and lowly differentiated liver cancer;
the melanoma is selected from the group consisting of multidrug-resistant melanoma, malignant melanoma, and combinations thereof;
the leukemia is selected from the group consisting of myeloid leukemia, T lymphocyte leukemia, and combinations thereof;
the lung cancer is selected from the group consisting of small cell lung cancer, non-small cell lung cancer, and combinations thereof;
the lymphoma is selected from the group consisting of B cell lymphoma, mononuclear cell lymphoma, T cell lymphoma, Non-Hodgkin's lymphoma, cutaneous T cell lymphoma, and combinations thereof;
the pancreatic cancer is selected from the group consisting of pancreatic ductal adenocarcinoma, liver metastatic pancreatic cancer, and combinations thereof; and/or
the kidney cancer is selected from the group consisting of kidney rhabdoid carcinoma, kidney smooth muscle carcinoma, kidney cell adenocarcinoma, and combinations thereof; and/or
the intestinal cancer is colorectal adenocarcinoma.

22. The method of claim 21, wherein the glioma is glioblastoma or glioblastoma multiforme;
the glioma comprises glioma with CDKN2A, PTEN and/or CDKN2C gene mutation;
the medulloblastoma is cerebellar medulloblastoma;
the breast ductal adenocarcinoma is invasive breast ductal adenocarcinoma;
the invasive breast ductal adenocarcinoma comprises invasive breast ductal adenocarcinoma with PTEN, RB1 and/or TP53 gene mutation;
the breast squamous cell carcinoma is breast acantholysis squamous cell carcinoma;
the metastatic breast cancer comprises breast cancer with CDH1 and/or PIK3CA gene mutation;
the liver cancer is anaplastic and lowly differentiated liver cancer;
the liver cancer comprises liver cancer with CTNNB1 and/or NRAS gene mutation;
the liver cancer is grade II-III/IV liver cancer;
the malignant melanoma comprises malignant melanoma with BRAF, CDKN2A and/or STK11 gene mutation;
the malignant melanoma is metastatic malignant melanoma;
the malignant melanoma is malignant melanoma with inguinal lymph node metastasis;
the myeloid leukemia is acute myeloid leukemia;
the T lymphocyte leukemia is acute T lymphocyte leukemia;
the pancreatic ductal adenocarcinoma comprises pancreatic ductal adenocarcinoma with TP53 gene mutation;
the kidney cell adenocarcinoma is metastatic kidney cell adenocarcinoma;
the kidney cell adenocarcinoma is primary kidney cell adenocarcinoma; and/or
the colorectal adenocarcinoma is selected from the group consisting of Dukes' type B colorectal adenocarcinoma, Dukes' type C, grade IV colorectal adenocarcinoma, and combinations thereof.

23. The method of claim 22, wherein the breast acantholysis squamous cell carcinoma is breast TNM IIB stage 2 primary acantholysis squamous cell carcinoma;
the breast acantholysis squamous cell carcinoma comprises breast acantholysis squamous cell carcinoma with CDKN2A, STK11, KDM6A and/or TP53 gene mutation;
the acute myeloid leukemia is M4 grade AML acute myeloid leukemia;
the acute myeloid leukemia is FAB M4 grade AML acute myeloid leukemia;
the FAB M4 grade acute myeloid leukemia comprises FAB M4 grade acute myeloid leukemia with NPM1 and/or DNMT3A R882C gene mutation; and/or
the Dukes' type C, grade IV colorectal adenocarcinoma comprises Dukes' type C, grade IV colorectal adenocarcinoma with CTNNB1, EGFR and/or FBXW7 gene mutation.

24. The method of claim 16, wherein the cancer is cancer with upregulation of the oxidative phosphorylation pathway of mitochondria and/or low activity of the mitochondria permeability transition pore in cancer cell.

25. The method of claim 24, wherein the upregulation of the oxidative phosphorylation pathway of mitochondria means that the ratio (E1/E0) of the level or expression E1 of the oxidative phosphorylation pathway of mitochondria in the cancer cell to the level or expression E0 of the oxidative phosphorylation pathway of mitochondria in the same type of cell is ≥1.2, preferably ≥1.5, more preferably ≥2, more preferably ≥3, more preferably ≥5; and/or
the low activity of the mitochondria permeability transition pore means that the ratio (A1/A0) of the activity level or expression level A1 of the mitochondria permeability transition pore in the cancer cell to the activity level or expression level A0 of the mitochondria permeability transition pore in the same type of cell is ≤0.8, preferably ≤0.7, more preferably ≤0.6, more preferably ≤0.5, more preferably ≤0.4, more preferably ≤0.3, more preferably ≤0.2, more preferably ≤0.1, most preferably ≤0.05.

26. The method of claim 16, wherein the pharmaceutically acceptable salt of the compound of formula I is a salt formed by the compound of formula I and an acid selected from the group consisting of hydrochloric acid, mucic acid, D-glucuronic acid, hydrobromic acid, hydrofluoric acid, sulfuric acid, nitric acid, phosphoric acid, formic acid, acetic acid, propionic acid, oxalic acid, malonic acid, succinic acid, fumaric acid, maleic acid, lactic acid, malic acid, tartaric acid, citric acid, picric acid, methanesulfonic acid , benzenemethanesulfonic acid, benzenesulfonic acid, aspartic acid, glutamic acid, and combinations thereof.

27. A method for enhancing the anticancer effect of anticancer drug, which comprises administering mitochondria permeability transition pore inhibitor to a subject in need.

28. The method of claim 16, wherein R₃ is hydroxyl, sulfhydryl, methoxyl, methylthio, or 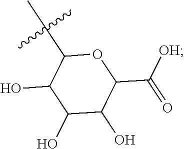 [SMILES: *C1OC(C(=O)O)C(O)C(O)C1O]
R₄ is hydrogen, halogen, —CN, hydroxyl, sulfhydryl, nitro, amino, —COOH, —CHO, substituted or unsubstituted C1-C6 alkyl, substituted or unsubstituted C3-C8 cycloalkyl, substituted or unsubstituted C1-C4 alkoxyl, substituted or unsubstituted C1-C4 alkylthio; and/or
R₅ is

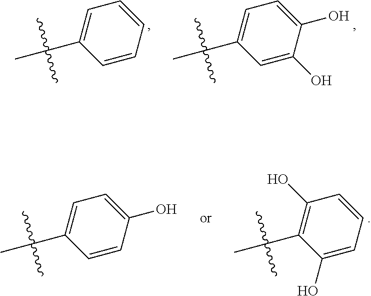 [SMILES: *c1ccccc1 ; *c1ccc(O)c(O)c1 ; *c1ccc(O)cc1 ; *c1c(O)cccc1O]

29. The method of claim 16, wherein the cancer is human cancer and non-human cancer;
the cancer is selected from the group consisting of adenocarcinoma, ductal carcinoma, squamous cell carcinoma, and combinations thereof;
the cancer is lowly differentiated, moderately differentiated or highly differentiated cancer cell;
the cancer is not sensitive to conventional radiotherapy and chemotherapy;
the cancer is recurrent or metastatic cancer; and/or
the cancer is cancer with stem cell properties.

30. The method of claim 16, wherein the cancer cell is selected from the group consisting of NCI-H82 cell, Daoy cell, Gp2D cell, U-937 cell, A-375 cell, SNU-398 cell, NCI-H1048 cell, HCC15 cell, ATN-1 cell, Jurkat, Clone E6-1 cell, MIA PaCa-2 cell, D283 Med cell, BT-549 cell, 22RV1 cell, H9 cell, G-401 cell, HCC1806 cell, OCI-LY-19 cell, MDA-MB-453 cell, SU-DHL-2 cell, G-402 cell, CCRF-CEM cell, HH cell, OCI-AML-3 cell, OCI-AML-4 cell, OCI-AML-5 cell, GAK cell, CHL-1 cell, NCI-H1155 cell, LS 180 cell, GB-1 cell, 786-O cell, SF126 cell, ACHN cell, COLO 320HSR cell, U-87 MG cell, WSU-DLCL2 cell, SNU-449 cell, C3A cell, NCI-H1793 cell, DU 145 cell, G-361 cell, and combinations thereof.

31. The method of claim 24, wherein the low activity of mitochondria permeability transition pore is made by administering the mitochondria permeability transition pore inhibitor.

32. The method of claim 31, the mitochondria permeability transition pore inhibitor is selected from the group consisting of Cyclosporin A, CyP-D protein inhibitor, peroxide scavenger, and combinations thereof.

33. The method of claim 27, wherein the anticancer drug is the compound of formula I, or an optical isomer thereof, or a racemate thereof, or a solvate thereof, or a pharmaceutically acceptable salt thereof of claim 16; and/or
the mitochondria permeability transition pore inhibitor is selected from the group consisting of Cyclosporin A, CyP-D protein inhibitor, peroxide scavenger, and combinations thereof.

34. A composition, the composition comprises:
(1) a first active ingredient, the first active ingredient is anticancer drug; and
(2) a second active ingredient, the second active ingredient is mitochondria permeability transition pore inhibitor.

35. The composition of claim 34, wherein the anticancer drug is the compound of formula I, or an optical isomer thereof, or a racemate thereof, or a solvate thereof, or a pharmaceutically acceptable salt thereof of claim 16; and/or
the mitochondria permeability transition pore inhibitor is selected from the group consisting of Cyclosporin A, CyP-D protein inhibitor, peroxide scavenger, and combinations thereof.
